(19) United States
(12) Patent Application Publication
(10) Pub. No.: US 2002/0193369 A1
(43) Pub. Date: Dec. 19, 2002

(54) Antifungal compounds and uses therefor

(76) Inventors: Penelope N. Markham, Oak Park, IL (US); Alexander A. Neyfakh, Chicago, IL (US); Yongzhi Xuan, Chicago, IL (US); David Crich, Chicago, IL (US); Mohammad-Rami Jaber, Romeoville, IL (US); Michael E. Johnson, Winnetka, IL (US); Debbie C. Mulhearn, Wheaton, IL (US)

Correspondence Address: Steven L. Highlander, FULBRIGHT & JAWORSKI L.L.P., SUITE 2400, 600 CONGRESS AVENUE, AUSTIN, TX, 78701, US

(21) Appl. No.: 10/008,375

(22) Filed: Nov. 2, 2001

Related U.S. Application Data
(60) Provisional application No. 60/245,548, filed on Nov. 2, 2000.

(51) Int. Cl. A61K 31/5415; A61K 31/538; A61K 31/496

(52) U.S. Cl. 514/224.2; 514/230.5; 514/254.07; 514/396

(57) ABSTRACT

The present invention provides compounds which, when used in combination with antifungal azoles, offer enhanced antifungal therapy. More particularly, these compositions (including carbazoles or triptycenes) can convert fungistatic drugs such as fluconazole into fungicidal drugs.

The government owns rights in the present invention pursuant to grant number AI 45271-01 from the National Institutes of Health. This application claims benefit of priority to U.S. Provisional Application Serial No. 60/24,55,48 filed Nov. 2, 2000, the entire contents of which are incorporated by reference.

### BACKGROUND OF THE INVENTION

1. Field of the Invention

The present invention relates generally to the fields of fungal disease and antimicrobial agents. More particularly, it concerns drug combinations that show enhanced antifungal activity.

2. Description of Related Art

The AIDS epidemic, advances in surgical procedures, and aggressive anticancer therapy have contributed to the surge of immunocompromised population. Coinciding with this surge is an increase in the incidence of clinically significant fungal infections (Dixon et al., 1996; Henderson and Hirvela, 1996). Candida albicans has become the fourth leading cause of nosocomial infections, with systemic candidiasis having a very high mortality rate, especially in newborns—up to 65% (Pacheco-Rias et al., 1997), and among cardiac surgery patients—up to 30% (Michaloupoulos et al., 1997). The majority of AIDS patients experience some form of candidiasis and many have to take antifungal drugs repeatedly, or even prophylactically on a daily basis. In the healthy population, more than half of all women experience at least one vaginal yeast infection, and about 8% suffer recurrent episodes. The morbidity, mortality and health care costs associated with fungal infections has commanded a need for effective antifungal agents.

Only a few classes of antifungal drugs are actively used in clinics. Flucytosine, a substituted pyrimidine, is converted by a fungi-specifc cytosine deaminase into 5-fluorouracil which causes the inhibition of DNA and protein synthesis. Due to frequent emergence of resistance, flucytosine is rarely used alone and is often co-administered with amphotericin B (Alexander & Perfect, 1997).

Amphotericin, a polyene antibiotic, has the broadest spectrum of activity of any available antifungal agent and is fungicidal when tested in vitro. It interacts with membrane sterols, alters membrane permeability and causes membrane leakage and death of the pathogen. However, amphotericin is toxic and has a very narrow therapeutic index. Even in therapeutic doses, it often causes severe side effects, including fevers, chills, nausea, vomiting, and nephrotoxicity (Brajtburg and Bolard, 1996).

Azole drugs, such as fluconazole, ketoconazole, and itraconazole, are much less toxic and have become drugs of choice for most indications. The primary target of azoles is the heme protein, lanosterol 14α-demethylase. By inhibiting this enzyme azoles prevent the synthesis of the major sterol of the fungal membrane, ergosterol, and cause accumulation of intermediate products (Kauffman and Carver, 1997).

The degree of the damage to fungal cells caused by the alterations of membrane sterols depends on the nature of the pathogen. While highly effective against Saccharomyces cerevisiae, azoles are less detrimental to Candida. They are not fungicidal toward the most common human fungal pathogen, C. albicans, and even their inhibitory effect on the growth of this yeast differs widely among different fungal isolates. While the growth of some isolates is strongly inhibited, the majority continue to grow even at very high concentrations of the drug with completely depleted ergosterol. This so-called post-MIC growth creates significant difficulties in determining the azole sensitivity of C. albicans isolates in clinical laboratories. g While the standards for determining minimal inhibitory concentrations (MIC) of most antimicrobial agents define MIC as the lowest concentration of the drug preventing any visible growth of a pathogen, the NCCLS standard for antifungal susceptibility testing (document M27-A) had to be formulated much less strictly and defines MIC as the lowest concentration of a drug causing 80% growth inhibition (NCCLS, 1997). The moderate inhibitory effect of azoles on the growth of C. albicans is also reflected in the phenomenon of “trailing endpoint” when the apparent MIC, or more correctly MIC₈₀, determined in broth microdilution tests shifts during the incubation (Rex et al., 1996; Revankar et al., 1998a). For most isolates the MIC of fluconazole lies below the clinically achievable 4 μg/ml if determined after 24 hours of incubation, but for many of them it exceeds 64 μg/ml after 48 hours.

The clinical effectiveness of azoles against C. albicans clearly exceeds their in vitro effectiveness. Indeed, isolates exhibiting a high rate of post-MIC growth in vitro were obtained from patients whose fungal infections were in fact later successfully treated with azole drugs (Revankar et al., 1998b). The reason for this discrepancy is that in the organism of a patient fungal infections are being suppressed not only by drug therapy but also by host defense mechanisms including phagocytes and antifungal immune response. Although merely slowing down the growth of the pathogen, azole drugs make it more susceptible to host defenses. Besides simply changing the dynamics of infection through growth inhibition, azoles have also been reported to make C. albicans cells more susceptible to phagocytes (De Brabander et al., 1980; Shigematsu et al., 1981).

In spite of the relative clinical success of azole drugs as compared to other antifungal agents, their inability to kill Candida cells without relying on host defense mechanisms is the likely reason for two highly undesirable clinical outcomes: recurrence of infection and development of azole resistance. As mentioned above, a significant percentage of women are suffering from recurrent vaginitis. In these cases azoles alleviate symptoms of infection but the infection relapses again a short time after treatment. The relapsed strain usually has the same sensitivity to the drug as the initial one (Fong et al., 1993; Lynch et al., 1996), thus suggesting that azole resistance is not the underlying cause of recurrence. Such host factors as immune deficiency, allergy, use of contraceptives, local pH, deficient production of IgA antibodies, and even psychological factors have been implicated in the phenomenon of recurrent vaginitis (White et al., 1997; Blasi et al., 1998; Irving et al., 1998; Kubota, 1998; Clancy et al., 1999). Importantly, however, molecular fingerprinting of the pathogen genome has shown that in more than 80% of cases the C. albicans strain which causes relapse is the same strain that caused initial infection (Schroppel et al., 1994; Fong, 1994, Vazquez et al., 1994; Lockhart et al., 1996). Similarly, recurrent azole-treated oropharyngial candidiasis which affects 50% of AIDS patients has been associated with re-growth of the same strain of C. albicans rather than with reinfection with other strains or development of azole resistance (Boerlin et al, 1996). It is highly likely, therefore, that many cases of recurrent candidiasis could have been prevented if azole drugs eradicated yeast cells rather than merely inhibited their growth.

Besides dramatically increasing the chances for the recurrence of infections, the survival of azole-treated Candida cells creates a breeding ground for the development of azole resistance. This resistance has become a serious clinical problem in recent years: its incidence is on the rise (Cameron et al., 1993; Redding et al, 1994; Revankar et al., 1998b), which endangers the future use of azole drugs in clinics. Clinical isolates of C. albicans demonstrate a number of biochemical mechanisms of resistance (reviewed in Sanglard et al., 1995; White et al., 1998;

Vanden Bossche et al., 1998). The first group of these mechanisms deals directly with the target of azole action, lanosterol 14α-demethylase. Point mutations in the gene of this enzyme, ERG11, alternatively called ERG16 or CYP51, over expression of this gene, or its amplification have been described in resistant clinical isolates of C. albicans. Additionally, azole-resistant C. albicans have been shown to over express multidrug efflux pumps: CDR1, CDR2, and MDR1. Expression of these membrane proteins leads to the decrease in the accumulation of azole drugs in the yeast cytoplasm and thus reduces their antifungal activity.

Importantly, each of these mechanisms individually provides relatively low level of azole resistance. Clinically resistant strains usually display a combination of resistance mechanisms described above. The development of these strains is a multi-step process in which genetic changes leading to resistance are accumulating gradually in response to selection with drugs (White, 1997; Franz et al., 1998; Franz et al., 1999; Lopez-Ribot et al., 1998; Cowen et al., 2000). The inability of azoles to kill yeast cells promotes this process. Indeed, a mutation leading to even a minor increase in the MIC of the drug gives mutated cells selective advantage over parental cells, so that they gradually overcome the yeast population infecting the patient. If azoles were fungicidal, both the parental cells and the cells with a slightly increased azole MIC would be eliminated, thus dramatically reducing chances for the development of resistant strains.

In summary, the clinical success of azole therapy of c. albicans infections is limited by the rather moderate inhibitory effect of ergosterol depletion on this pathogen. Large pharmaceutical companies are tying to improve the effectiveness of antifungal therapy by identifying alternative drugs attacking new molecular targets of the pathogen. As of yet, these extensive screening programs have not yielded a drug with an activity significantly exceeding that of azoles. An alternative approach to drug discovery has been utilized previously by the inventors, namely, the identification of potentiators of existing antimicrobial agents. In particular, in this bacterial work, the inventors have identified a number of potentiators of fluoroquinolone antibiotics, which act by inhibiting multidrug-efflux transporters of pathogenic Gram-positive cocci (Markham et al., 1999). More recently the inventors identified compounds which, when combined with bacteriostatic antibiotics, exert bactericidal effect. With respect to antifungal agents, the inventors embarked on finding a compound that would similarly potentiate the antifungal effect of azoles, the most effective and popular antifungal drugs to date.

### SUMMARY OF THE INVENTION

Thus, in one embodiment of the invention, there is provided a method for enhancing the antifungal action of azoles comprising contacting a fungal cell and an azole with a second agent comprising a carbazole, a triptycene, a triphenyl, a compound, or a pharmaceutically acceptable salt or hydrate thereof, having the structure of formula (I′):

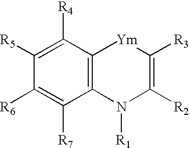

whereinY is S or O; m is 0 or 1; R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)CX₃, (—CH₂)ₙCHOHCX₃, 3-(4-styryl-piperazin-1-yl)propan-2-ol, allyl, benzyl, H, or an alkyl chain up to 4 carbons; X is a halogen; R₈ is a H, or an alkyl chain up to 4 carbons; n is 0, 1, 2, 3, 4, or 5; R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; R₃ is a hydrogen, a halogen, acetamido, amino, pyridyl, a halo substituted pyridyl, a pyridyl salt, benzyl, 1- or 2-napthyl, styryl, phenethyl, faranyl, phenyl, a branched or straight-chained alkyl group up to 4 carbons, an ortho, meta, or para toluoyl, or a halo- or acetamido-substituted phenyl group; R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons; or a compound, or a pharmaceutically acceptable salt or hydrate thereof, having the structure of formula (I″):

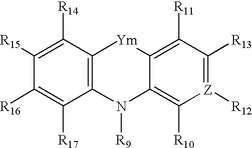

wherein Y is S or O; m is 0 or 1; Z is N or C; R₉ is an —(CH₂)ₙCO₂R₁₈, —(CH₂) ₙCHO, —(CH₂)ₙOH, —(CH₂)ₙCHOHCH₃, (CH₂)ₙCHOHCH₂N(R₁₈)₂, —(CH₂)ₙ CHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein X is a halogen; R₁₈ is a H, or an alkyl chain up to 4 carbons; n is 0, 1, 2, 3, 4, or 5; R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons, with the proviso that when Z is N, R₁₂ is nonexistent; or a compound, or a pharmaceutically acceptable salt or hydrate thereof, having the structure of formula (I′″):

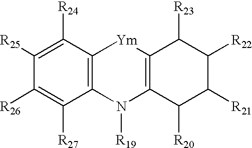

whereinY is a S or O; m is 0 or 1; R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃—(CH₂)ₙCHOHCX₃, allyl, benzyl, H, or an alkyl chain up to 4 carbons; X is a halogen; R₂₈ is a H, or any alkyl chain up to 4 carbons; n is 0, 1, 2, 3, 4, or 5; R₂₀, R₂₁ are a hydrogen, or are part of a benzene ring; R₂₂, R₂₃ are a hydrogen, or are part of a benzene ring; R₂₄, R₂₅, R₂₆, R₂₇ are hydrogen, any halogen, trifluoromethyl, thioalkyl, alkoxy, alkylamido, phenyl, or a branched or straight-chained alkyl group up to 4 carbons.

The method for enhancing the antifungal action of azoles could include the second agents comprising an indole, a 1,4-dihydroquinolin, a pyridino[4,3-b]indole, a 5,6,7,8,9,10-hexahydroacridine, or a 5,10-dihydropydidino[3,4-b]quinoline, and any combinations of these second agents, or derivatives of these agents.

The method may include a second agent, or a pharmaceutically acceptable salt or hydrate thereof, which has the structure of formula (I′), as shown previously, wherein Y is S or O; m is 0 or 1; R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX ₃, (—CH₂)ₙCHOHCX₃, 3-(4-styryl-piperazin-1-yl)propan-2-ol, allyl, benzyl, H, or an alkyl chain up to 4 carbons; X is a halogen; R₈ is a H, or an alkyl chain up to 4 carbons; n is 0, 1, 2, 3, 4, or 5; R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, orpara toluoyl, or a halo- or acetamido-substituted phenyl group; R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons.

A specific example of the structure of formula (I′) would have m=0; R₁ is a H or —CH₂CO₂H; R₂ is a phenyl, 2-furanyl, m-fluorophenyl, or a pyridyl salt; R₄, R₅, R₆ are a H, Cl, or Br; and R₃, R₇ are a H.

In another specific example, the structure of formula (I′) would have m=0; R₂ is a phenyl, m-toluoyl, or m-fluorophenyl; R₅ is a Cl or Br; and R₁, R₃, R₄, R₆, R₇ are a H.

The method may include a second agent, or a pharmaceutically acceptable salt or hydrate thereof, having the structure of formula (I″), as shown previously, wherein Y is S or O; m is 0 or 1; Z is N or C; R₉ is an —(CH₂)ₙCO₂R₁₈, —(CH₂)ₙCHO, —(CH₂)ₙOH, —(CH₂)ₙCHOHCH₃, (CH₂)ₙCHOHCH₂N(R₁₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; X is a halogen; R₁₈ is a H, or an alkyl chain up to 4 carbons; n is 0, 1, 2, 3, 4, or 5; R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino alkylamido, carbamyl alkyl ester, phenyl, or a branched or straight-chained alkyl group up to 4 carbons, with the proviso that when Z is N, R₁₂ is nonexistent.

A specific example of the structure of formula (I″) would have Y as a S; m=0 or 1; Z is a C; R₉ is a 3-(4-styryl-piperazin-1-yl)propan-2-ol, —(CH₂)ₙOH, or —(CH₂)ₙCHOHCH₃; n=1, 2, or 3; R₁₃, R₁₅ are a H, Cl, I, or Me; and R₁₀, R₁₂, R₁₁, R₁₄, R₁₆, R₁₇ are a H.

In particular, the structure of formula (I″) could have m=0; Z is a C; R₉ is a —(CH₂)ₙCO₂R₁₈, —(CH₂)ₙCHO, —(CH₂)ₙCHOHCH₂OH; R₁₈ is a H; n=1, 2, or 3; R₁₃, R₁₅ is a Cl, Br, I, or H; and R₁₀, R₁₂, R₁₁, R₁₄, R₁₆, R₁₇ are a H.

The method may include a second agent, or a pharmaceutically acceptable salt or hydrate thereof, that has the structure of formula (I′″), as shown previously, wherein Y is a S or O; m is 0 or 1; R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons; X is a halogen; R₂₈ is a H, or any alkyl chain up to 4 carbons; n is 0, 1, 2, 3, 4, or 5; R₂₀, R₂₁ are a hydrogen, or are part of a benzene ring; R₂₂, R₂₃ are a hydrogen, or are part of a benzene ring; and R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

A specific example of the structure of formula (I′″) would have m is 0; R₁₉ is a H, —(CH₂)ₙCO₂H, or —(CH₂)ₙOH; n is 1 or 2; R₂₂, R₂₃ are part of a benzene ring; R₂₅ is a Cl, or I; and R₂₀, R₂₁, R₂₄, R₂₆, R₂₇ are a H.

The method may include second agent, wherein the second agent is a carbazole,or a pharmaceutically acceptable salt or hydrate thereof, having the formula (I):

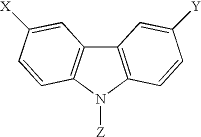

wherein X is an H, or a chloro, bromo, iodo, hydroxy, methoxy, amino, nitro, or dimethylamino; Y is an H, or a chloro, bromo, iodo, hydroxy, methoxy, amino, nitro, or dimethylamino; and Z is —(CH₂)ₙCO₂H, —CH(OH)CH₃, —(CH₂)ₙCHOHCH₂OH, —(CH₂)ₙCH₂OH, —(CH₂)ₙC(═O)(CH₂)ₘCH₃, or —(CH₂)ₙCO₂(CH₂)ₘCH₃, wherein n and m are independently 0, 1, 2, 3, or Z is 4, 3-(4-styryl-piperazin-1-yl)propan-2-ol, an alkyl amide, or an alkoxyl.

A specific examples of the structure of formula (I) would have X and Y as halogens; X and Y are the same; X and Y are different; or Z comprises 3 carbon atoms.

The method may include the second agent, wherein the second agent is a triptycene, or a pharmaceutically acceptable salt or hydrate thereof, having the formula (II′):

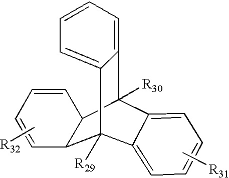

wherein R₂₉, R₃₀, R₃₁, R₃₂ are a H, —(CH₂)ₙCO₂R₃₃, —(CH₂)ₙOH, —(CH₂)ₙCHO, —(CH₂)ₙC(═O)CX₃, —(CH₂)ₙC(═O)Cl, —(CH₂)ₙC(═O)R₃₃, —(CH₂)ₙCHXR₃₃, —(CH₂)ₙCX₂R₃₃, —(CH₂)ₙN(R₃₃)₂, —(CH₂)ₙCH(OH)CH₃, —(CH₂)ₙCH(OH)CH₂OH, —CH═CHCH═CHCO₂R₃₃, —CH₂CH═CHCO₂R₃₃, —(CH₂)ₙCX₃, —C≡CCOR₄, —CON(R₃₃)₂, [1,3]-dioxolan-2-yl, phosphoryl dibenzylester, phosphoryl, sulfuryl, allyl, ethylene, nitro, or a halogen; wherein X is a halogen; R₃₃ is a H, or any alkyl chain up to 4 carbons; and n=0, 1, 2, 3, 4, or 5.

A specific examples of the structure of formula (II′) would have R₂₉ as a —(CH₂)ₙCO₂R₃₃, —(CH₂)ₙOH, or —(CH₂)ₙCHO, wherein n=0, 2, 3, or 4; R₃₃ is H; and R₃₀, R₃₁, R₃₂, are a H.

Furthermore the method for enhancing the antifungal action of azoles could include the second agent, wherein the second agent is a triptycene,or a pharmaceutically acceptable salt or hydrate thereof, having the formula (II):

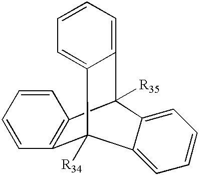

Wherein R₃₄ is a —CO₂H, —CH₂OH, —CHO, —C(═O)Cl, —(CH₂)₃OCH₃, —(CH₂) ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; or branched hydroxyalkyl, branched alkoxyl, alkyl amide, or branched fluoro-, chloro-, bromo-, or iodo-alkyl; R₃₅ is an H, or —CO₂H, —CH₂OH, —CHO, —C(═O)Cl, —(CH₂)₃OCH₃, —(CH₂)ₙOH, —(CH₂)ₙC(═O)CH₃, —(CH₂)ₙ CO₂Me, —(CH₂)ₙC(═O)(CH₂)ₘCH₃, —(CH₂)ₙCO₂(CH₂)ₘCH₃, —(CH₂) ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; branched hydroxyalkyl, alkyl amide, branched alkoxyl, or branched fluoro-, chloro-, bromo-, or iodo-alkyl.

Specific examples of formula (II) would have R₃₄ and R₃₅ being different; R₃₅ as a H; R₃₄ as a halogen orR₃₄ comprising two carbons.

In the fungal cell may be selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria, Aspergillus, and Candida albicans. The method can be applied to fungus that is resistant to azole treatment.

The azole may be chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-975 1, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

The method may further comprising contacting the fungal cell and the azole and at least two distinct second agents.

The method could also comprise of an azole that is fungistatic in the absence of the second agent, and fungicidal in the presence of the second agent.

Furthermore the method for enhancing the antifungal action of azoles could include the second agent, wherein the second agent is a triphenyl, or a pharmaceutically acceptable salt or hydrate thereof, having the formula (III):

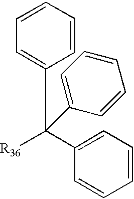

wherein R₃₆ is a H, —(CH₂)ₙCO₂R₃₇, —(CH₂)ₙOH, —(CH₂)ₙCHO, —(CH₂)ₙC(═O)CX₃, —(CH₂)ₙC(═O)Cl, —(CH₂)ₙN(R₃₇)₂, —(CH₂)ₙCH(OH)CH₃, —(CH₂)ₙ CH(OH)CH₂OH, —CH═CHCH═CHCO₂R₃₇, —CH₂CH═CHCO₂R₃₇, —(CH₂)ₙCX₃, —C≡CCOR₃₇, —CON(R₃₇)₂, [1,3]-dioxolan-2-yl, phosphoryl dibenzylester, phosphoryl, sulfuryl, allyl, ethylene, nitro, or a halo; wherein X is a halogen; R₃₇ is a H, or any alkyl chain up to 4 carbons; and n=0, 1, 2, 3, 4, or 5.

A specific example of the structure of formula (III) would have R₃₆ is a —(CH₂)ₙCO₂H or —(CH₂)ₙCHO; and n=2.

In another embodiment, there is provided a method of treating a fungal infection in a subject comprising administering to the subject, in antifungal amounts, an azole and a second agent comprising a triptycene, a triphenyl, or a compound having the structure of formulas I, I′, I″, I′″, II, II′ or III, as defined above. The azole may be chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869. The fungus may be selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus. The fungus may be resistant to azole treatment. The second agent may be with, or separate from, the azole. If separate, they are delivered with 2 hours of each other. Routes include intravenous delivery. One or both of the azole and the second agent may be administered repeatedly. The subject may be an animal, e.g., a human. The method may further comprise administering at least two distinct second agents to the subject. The azole is may be fungistatic in the absence of the second agent, and fungicidal in the presence of the second agent.

In still another embodiment, there is provide a method of screening for potentiators of azole antifungal activity comprising (a) providing a fungal cell; (b) contacting the cell with an azole and a candidate potentiator substance; and (c) comparing the antifungal activity the azole with the antifungal activity of the azole in the absence of the candidate potentiator substance. The method may further comprise comparing the antifungal activity of the candidate potentiator substance in the absence of the azole. The fungal cell may be selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus. The fungal cell may be resistant to azole treatment. The azole may be chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869. The azole may be fungistatic in the absence of the second agent, and fungicidal in the presence of the second agent.

In still another embodiment, there is provided a pharmaceutical composition comprising an azole and a second agent comprising a triptycene, a triphenyl, or a compound having the structure of formulas I, I′, I″, I′″, II, II′ or III, as defined above. The azole may be chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

In still yet another embodiment, there is provide a method for suppressing the emergence of azole resistance in fungi comprising contacting a fungal cell during the course of azole therapy with a second agent comprising a triptycene, a triphenyl, or a compound having the structure of formulas I, I′, I″, I′″, II, II′ or III, as defined above. The azole may be chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869. The method may further comprise contacting the fungal cell with at least two second agents. The fungal cell may be selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus. Contacting the fungal cell with a second agent can occur before, during, or after azole administration.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1—Structures of Lead Compounds INF 799, INF 800, INF 801 and INF 802 and other specific compounds.

FIG. 2—Effects of Fluconazole, INF 801, INF 802, and Combinations Thereof. Plates photographed at 2 days after plating.

FIG. 3—Killing Effect of the Combinations of Fluconazole with the Lead Compounds.

Fluconazole (Flu), INF 801 and INF 802 were added 32, 10 and 10 μg/ml, respectively.

FIG. 4—TLC Analysis of Sterols of C. albicans. Treatment was performed for 24 hours with differing concentrations of fluconazole in the presence and absence of INF 801. Flu =fluconazole; Erg=ergosterol.

### DETAILED DESCRIPTION

I. The Present Invention

As discussed above, azoles are an important drug in the treatment of fungal disease. However, also mentioned above are the limitations seen in the clinical use of azoles. The inventors hypothesized that azoles, by depleting membrane ergosterol, change the biochemistry of fungal cells by altering their permeability properties, thus rendering them susceptible to Aid otherwise harmless compounds. This hypothesis is supported by earlier results obtained by others, which showed that post-MIC growth of C. albicans could be inhibited by low pH (Marr et al., 1999), or the presence of high concentrations of acetate ions in the medium (Shimokawa and Nakayama, 1999). Furthermore, treatment with an azole drug has been shown to make C. albicans susceptible to killing mediated by hydrogen peroxide (Shimokawa and Nakayama, 1992).

Recently it has been reported that cyclosporine A is synergistically fungicidal in vitro in combination with fluconazole towards C. albicans (Marchetti et al., 2000a). Using very high doses of fluconazole, and higher than recommended doses of cyclosporine A, the same group demonstrated increased cidal activity of this combination in an in vivo model (Marchetti et al., 2000b), demonstrating proof of concept for the approach of combining fluconazole with a fungicidal potentiator.

Although none of these findings could have clinical applications, they raised the possibility of identifying a compound that would inhibit post-MIC growth, or even be fungicidal after azole-mediated depletion of ergosterol. The inventors have established the feasibility of developing such a potentiator and, in fact, have identified several groups of azole potentiators, including compounds completely inhibiting post-MIC growth and reversing some of the azole-resistance mechanisms. Two of the identified compounds show the most desired property, i.e., strong fungicidal activity when used in combination with azoles.

II. Target Fungi and Their Related Pathologies

In the United States, blastomycosis, coccidioidomycosis and histoplasmosis are the major causes of systemic mycotic infection in normal human hosts. Sporotrichosis is a fourth invasive fungal disease, but occurs with broader distribution than the previous three. A variety of other fungal agents, including Candida and Aspergillus species, can colonize the mucocutaneous surfaces of normal human hosts, but rarely cause disease. Much more typical are fungal infections in immune-compromised individuals.

Blastomyces—Blastomycosis is a systemic mycotic infection that is cause by the dimorphic fungus Blastomyces dermatitidis. The initial portal of entry is the respiratory tract, with inhaled organisms deposited in the peripheral air spaces of the lower lobes. Hematogenous dissemination with metastatic spread to a variety of sites, particularly the skin, skeletal system, genitalia and central nervous system may occur. The pathologic hallmark is mixed acute and chronic inflammation. Treatment generally involves amphotericin B, given at a total dosage of 2.5 to 3.0 grams over 2 to 3 months. Fluconazole (400 mg/day) and itraconazole (400-800 mg/day) also have been employed more recently.

Histoplasma—Histoplasmosis, a systemic mycosis characterized by infection of the fixed and circulating phagocytic cells of the reticuloendothelial system, is caused by the dimorphic fungus Histoplasma capsulatum. The fungus grows in many parts of the world, particularly in soil enriched with the fecal material of birds or bats. Typically infection occurs when the soil is disturbed, causing an aerosol-type infection. Regional spreading to lymph nodes and bloodstream occurs rapidly. One to three weeks after infection, necrotizing granulomatous responses develop. Inteferon-gamma and IL-12 appear to be of great importance in the immune defense against the disease. Typical treatment is with amphotericin B, in a total dose of between 500 and 1000 mg. Azoles also are suitable therapies.

Coccidioides—The causative agent for coccidioidomycosis is the dimorphic fungus Coccidioides immitis. It can exist as a non-invasive saprophyte on tissue surfaces, but inhalation of the arthrospores results in production of mature spherules, the definitive tissue pathogen. The natural habitat of the disease is in the lower Sonoran life zone, but transmission is so efficient, the disease may spread many miles away. In some endemic region, infection is virtually universal. Cell mediated immunity is critical to controlling the infection, and immune-suppressed individuals show reduced granuloma formation, and concomitant increase spherule burden. Amphotericin B, fluconazole and itraconazole all are used in treatment.

Sporothorix—Sporothorix schenckii is a dimorphic fungus found in both tropical and temperate climates. Disease commonly arises from subcutaneous inoculation with infections spores by a contaminated thorn or other sharp object. In rare cases, spores may be inhaled. Following subcutaneous implantation, pseudoepitheliomatous hyperplasia of the overlying layers of the skin develop, producing a verrucous, sometimes ulcerating lesion. From this initial site, there is slow spread along the draining lymphatics, and secondary skin lesions. Amphotericin B is the preferred treatment.

Candida—Candidiasis comprises clinical infections that are caused by different dimorphic fungi of the genus Candida. The most virulent are C. albicans and C. tropicalis, but a C. krusei, C. parapsilosis and C. guilliermondii can cause disease in immunocompromised patients. Candida species are part of the normal GI flora in 50% of persons, and in vaginal flora in 20% of non-pregnant women. Overgrowth remains trivial unless the mucocutaneous surfaces are penetrated.

Variations on Candida pathology include mucosal candidiasis, cutaneous candidiasis, chronic mucocutaneous candidiasis, candidal peritonitis, candidal endocarditis, pulmonary candidiasis, urinary tract candidiasis, and disseminated candidiasis. Diagnosis is by microscopic examination and culture. Amphotericin B is the standard therapy, with a total dose of 500 to 1000 mg. Treatment typically involves mystatin, clotrimazole or miconazole for minor cutaneous or vaginal candidiasis. Fluconazole or itraconazole at 400 to 800 mg/day also may be used.

Aspergillus—Aspergillosis covers a group of different illnesses that have a major impact on the lungs, and are caused by dimorphic fungi of the genus Aspergillus. A single species, Aspergillus fumigatus, accounts for one-half to two-thirds or of all clinical disease caused by Aspergillus, with Aspergillus flavus accounting for most of the remainder. Aspergillus is almost always transmitted through the air, and it implants in the lungs, nasal sinuses, palate, and epiglottis. The most serious form of aspergillosis is found severely immunocompromised patients, characterized by necrotizing bronchopneumonia. Therapy usually involves amphotericin B, with possible surgical ablation. Flucytosine or rifampin often is added to the regimen. Azoles may be used as end stage “wrap-up” treatment.

Other significant fungal infections are caused by Cryptococcus, Torulopsis, Paracoccidioides, Rhizopus, Mucor and Absidia species.

III. Antifungal Azoles

The present invention relies, in part, on the use of antifungal azoles. These include the 1; imidazoles and the N-substituted triazoles. While more of the former are currently in use, more recent efforts have focused on the triazoles given their more slow metabolism and the lesser effect on human sterol synthesis. Currently used imidazoles include chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole and tioconazole, UR-9746, UR-9751 vibunazole. Fluconazole, terconazole, genaconazole, itraconazole, voriconazole, posaconazole, ravuconazole, parconazole, SS 750, R120758, T-8581, TAK 456, TAK 457, BMS 207147, R-102557, SYN 2869 and other triazoles.

At concentrations achieved during systemic administration, the major effect of azoles on fungi is in the inhibition of sterol 14-α-demethylase, a microsomal cytochrome P₄₅₀-dependent enzyme system. The inhibition of this enzyme leads to reduced ergosterol synthesis and an excess of 14-α-methyl sterols. This sterol build-up is thought to disrupt the close packing of acyl chains of phospholipids and impair the function of some membrane-bound enzyme systems.

Fluconazole: Fluconazole is a fluorinated bis-triazole. It is almost completely absorbed from the GI tract. Concentrations in plasma are essentially the same when the drug is given orally or intravenously, and bioavailability is not altered by food or gastric activity. Human adult dosages are in the range of 50 to 400 mg daily, with both oral and intravenous formulations available.

Ketoconazole: Ketoconazole is administered orally and is used to treat a number of superficial and systemic fungal infections. Oral absorption varies between individuals. Simultaneous administration of H₂ histaminergic receptor blocking agents and antacids may limit bioavailability. Oral doses range from 200-800 mg, giving peak plasma concentrations of 4-20 μg/ml.

Miconazole: Miconazole is a close relative of econazole. It readily penetrates the strateum corneum and persists for more than 4 days after application. Less than 1% is absorbed from the blood. It is available as a 2% dermatologic cream, spray, powder or lotion, 100 and 200 mg suppositories (7 day or 3 day regimen, respectively).

Itraconazole: Itraconazole is a triazole closely related to ketoconazole. Absorption in the fasting state is 30% of that when the drug is take with food. Although concentrations of this drug in plasma are much lower than with the same doses of ketoconazole, tissue concentrations are high. Concurrent administration of rifampin decreases concentrations of itraconazole in plasma substantially. Typical oral dose for adults is 200 mg once daily, but higher doses may be used for limited duration.

Clotrimazole: Clotrimazole is a topical antifungal. Absorption is less than 0.5% after application to the skin, but 3-10% from the vagina. Typical dosage is as a 1% cream lotion or solution. It also is used in 100 or 500 mg vaginal tablets and 10 mg troches. Skin applications are twice a day; vaginal regimens include one 100 mg tablet per day for 7 days, the 500 mg tablet used once, or 5 g cream for 7-14 days.

Econazole: Econazole is the deschloro derivative of miconazole. It readily the penetrates the stratum corneum and is found in effective concentrations down to the mid-dermis. Less than 1% appears to be absorbed into the blood. It is provided in a 1% cream applied twice per day.

Terconazole: Terconazole is a ketal triazole with structural similarity to ketoconazole. It is available as an 80 mg suppository inserted vaginally at bedtime for three days, or as a 0.4% vaginal cream used for 7 days.

Butoconazole: Butoconazole is comparable to clotrimazole and is available as a 2% vaginal cream. Typical treatment regimen is once a day application for three days.

Oxiconazole: Oxiconazole is a topical antifungal for treatment of common pathogenic dermatophytes. It is available in a 1% cream.

Sulconazole: Oxiconazole is a topical antifungal for treatment of common pathogenic dermatophytes. It is available in a 1% solution.

Other azoles include chlormidazole, isoconazole, bifonazole, democonazole, fenticonazole, lanoconazole, lombazole, sertaconazole and genaconazole. Voriconazole, posaconazole, ravuconazole, parconazole, UR-9746, UR-9751, T-8581 (Yotsuji et al., 1997), BMS 207147 (Fung-Tomc et al., 1999), SS 750 (Takeda et al., 2000), TAK 456, TAK 457, R-102557 (Oida et al., 2000), UR-9751, R-120758 (Kamai et al., 2000), and SYN 2869 (Johnson et al., 1999) are under development.

IV. Azole Potentiators

In one aspect of the invention, there is provided a series of compounds that enhance the activity of antifungal azoles. In particular, these potentiators can convert azoles, which are normally only fungistatic, to fungicidal compounds. The potentiators can also possess antifungal activity when used alone.

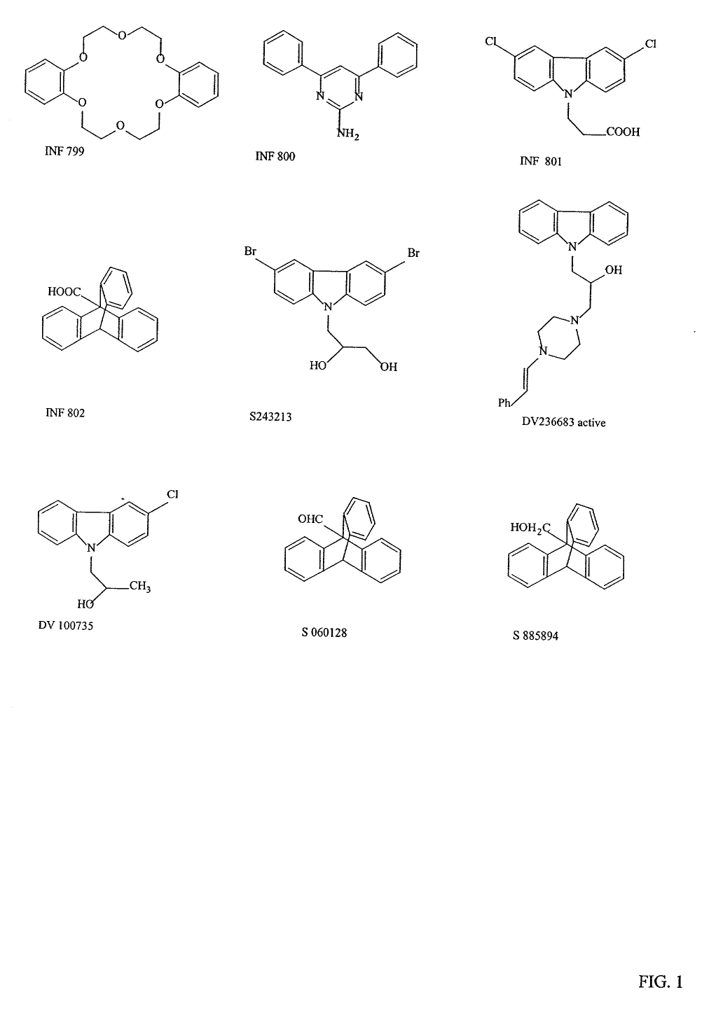

Lead Compounds: By screening a chemical library, the inventors identified 13 compounds that strongly inhibited post-MIC growth of C. albiacans but had no effect on yeast viability and did not act on azole-resistant isolates. The most potent of these compounds was INF 799 (FIG. 1), a crown ether, which was active at a concentration as low as 2 μg/ml. When added at this or higher concentrations to the cultures of C. albicans (ATCC 36801, 90028, and nine azole-sensitive clinical isolates) growing with 16 μg/ml of fluconazole, INF 799 inhibited growth by approximately six-fold. At the same time, without fluconazole present, it had no effect on the growth of C. albicans even at a concentration as high as 80 μg/ml. The MIC₈₀ of fluconazole was also not affected by this compound or any other compound of this group either in the sensitive, or in azole-resistant isolates. It appears, therefore, that INF 799 and other members of this group of lead compounds specifically inhibit growth of the ergosterol-depleted yeast.

Compound INF 800 (FIG. 1), at the concentration of 1 μg/ml and higher, in addition to inhibiting post-MIC growth of C. albicans, reduced the MIC₈₀ of fluconazole in sensitive isolates by two-four fold: i.e., from 0.5-1.0 μg/ml to 0.125-0.25 μg/ml. In some of resistant isolates its effect was even more pronounced. In particular, the MIC of the isolate #4 (White, 1997) was reduced by this compound from 8 to 0.25 μg/ml. This isolate has been thoroughly characterized and found to have only one genetic change associated with azole resistance—over expression of the MDR1 transporter gene (White, 1997). It appears, therefore, that INF 800 has a dual activity. Like the majority of the identified leads, it inhibits the growth of ergosterol-depleted cells and, additionally, inhibits MDR1-mediated azole resistance. Its moderate effect of the MIC of sensitive isolates is likely due to the inhibition of the MDR1 transporter expressed at the normal level.

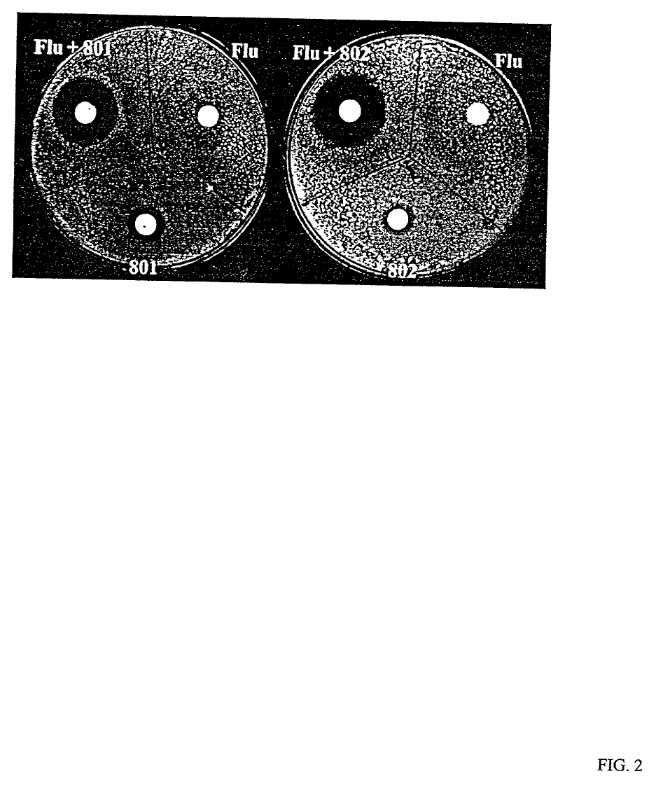

The activity of compounds INF 801 and 802 is best illustrated in FIG. 2. Here, paper disks soaked in solutions of fluconazole (1 mg/ml), INF 801, INF 802 (both at 2.5 mg/ml in DMSO; DMSO controls showed no effect on fungal growth), or the combinations of fluconazole with these compounds were placed on the surface of YPD agar plates inoculated with C. albicans. After 24 hours of incubation fluconazole produced a pronounced zone of growth inhibition, but later the slowly growing ergosterol-depleted C. albicans largely caught up with the rest of the plate. These zones of inhibition can still be noticed in FIG. 2. Compounds INF 801 and INF 802 produced narrow zones of inhibition, indicating that they are toxic for Candida cells only at high concentrations. In fact, in liquid culture compound INF 801 inhibited growth of C. albicans only at concentrations exceeding 30 μg/ml whereas INF 802 did not affect the growth of the yeast even at 80 μg/ml, the limit of its solubility. In contrast to individual compounds, the combinations of fluconazole with INF 801 or INF 802 produced large zones of inhibition lacking any growth of C. albicans (FIG. 2). Most remarkably, no growth of Candida cells was detected within these zones even after a week of incubation.

Analogs of INF 801 (Carbazoles): Using a small set of commercially available analogs, several carbazoles, at a concentration of 10 μg/ml or less, showed the ability to synergize with fluconazole (32 μg/ml) and result in a fungicidal effect on C. albicans. These and their inactive cogeners are shown below.

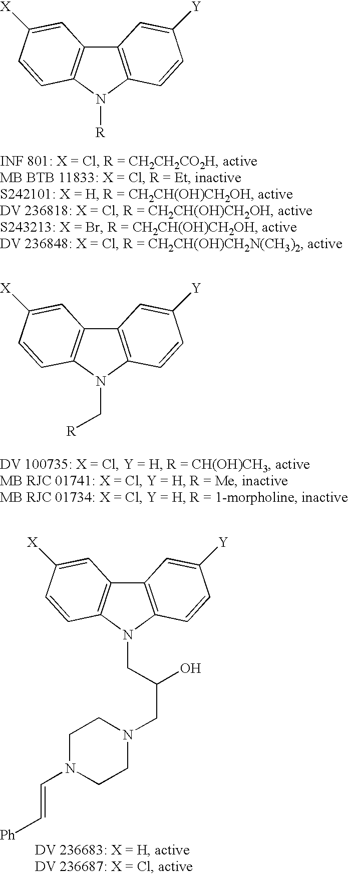

From the first block of compounds the inventors deduced primarily that the presence of a hydrogen bond donor on a substituent attached to the carbazole nitrogen (9-position) is important. From the comparison of the activity of S243213 (0.6 μg/ml), DV 236818 (5 μg/ml) and S242101 (12.5 μg/ml) it was inferred that, for a given N-substituent, 3,6-dibromo is better than 3,6-dichloro which is better than the corresponding non-halogenated carbazole. A congener with a single halogen bearing the same N-substituent as the active carbazoles was unavailable for testing. However, from DV 100735 it was apparent that a single halogen is probably sufficient. The activities of the final carbazoles illustrated, DV 236683 (5 μg/ml) and DV 236687 (10 μg/ml) show how, if the N-substituent is of sufficient size, the halogens on the ring are not a major factor in determining activity. It was noted that several indoles substituted on nitrogen with the type of groups that conferred activity in the carbazole series were not active, suggesting that the third ring is necessary.

As a result of this analysis the inventors prepared a series of rational structural modifications of N- substituted carbazoles. Initially, the inventors began by taking INF 801 as a lead and optimizing the substituents in the 3, 6-positions. Fortuitously, electrophilic aromatic substitution of readily available carbazole led directly to the 3,6-disubstituted series (Sundberg, 1984) with no significant problems. Moreover, carbazoles 1-6 are all known compounds, and the well-described straightforward preparations of 1, 2, 5 and 6 (Tucker, 1926; Grotta et al., 1964; Stepanova and Shishkina, 1967; Gaber, 1995) were followed. Preparation of the tetramethyl analog 7 of 6, yet to be prepared, should not be problematic and will involve alkylation of the more acidic carbazole N-9 with sodium hydride and benzyl bromide to give 3, 6-diamino 9-benzylcarbazole. This will be followed by introduction of the four methyl groups on the remaining nitrogens by means of the well known Borch reductive alkylation (Borch, 1988), and finally hydrogenolysis of the N-benzyl group. Each of these carbazoles will then be subjected to conjugate addition (Perlmutter, 1992) with acrylic acid to give directly 8-14.

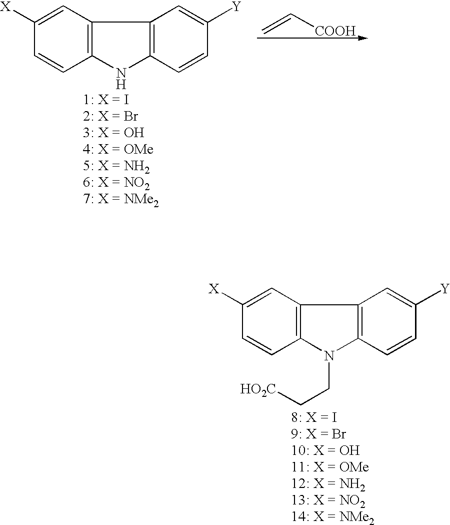

With the best substituent at the 3, 6-position the inventors proceeded to optimization of the N-9 chain. By way of example the inventors used the 3, 6-dibromide (2) below to illustrate the type of derivatives that were constructed. The inventors incrementally increased the length of the chain between the carbazole nitrogen and a series of polar head groups. 3, 6-Dibromo-N-carboxymethylcarbazole (15) was obtained by alkylation of 2 with methyl bromoacetate followed by saponification. The first homolog 9 can be obtained by N-alkylation of 2 with allyl bromide followed by hydroboration/oxidation as illustrated for homolog 16 below. That gave access to the corresponding primary alcohol 17 which was converted to acid 18, esters, etc., by routine techniques. Dihydroxylation of the alkene with osmium tetroxide according to the catalytic van Rheenan protocol (Van Rheenan et al, 1988) provided the homolog 19 of the initial lead DV236818, whereas oxymercuration, demercuration and oxidation will yield ketones 20. A list of specifically contemplated carbazole derivatives is shown in Table 1. Other polar substituents at the terminus of the side chain, whose nature will be determined following testing of the compounds illustrated, also will be explored in the future. Higher homologs will be obtained by N-alkylation with the appropriate unsaturated alkyl bromides, which are commercially available, and the same scheme will be followed.

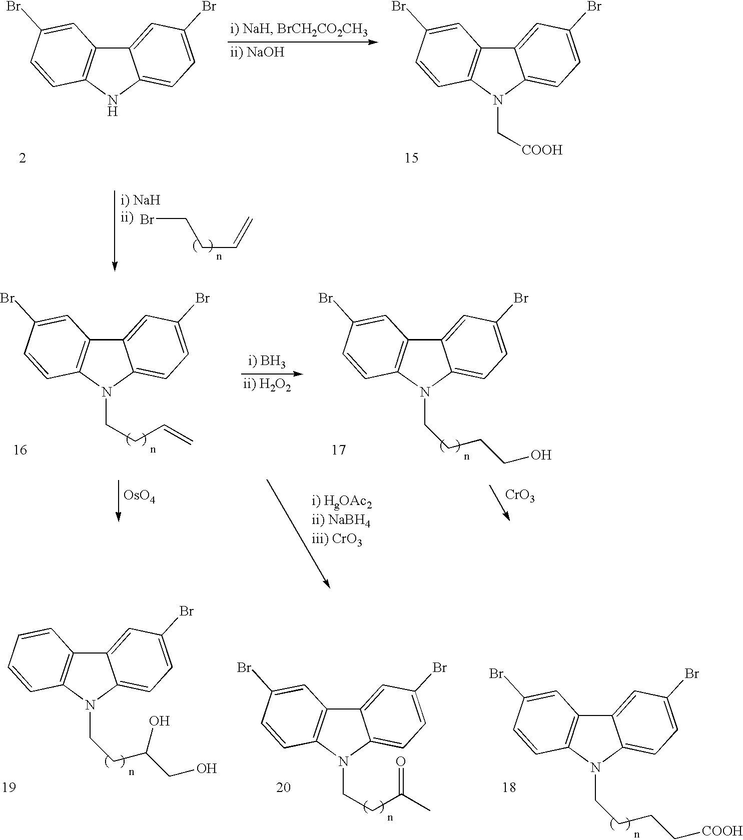

Finally, with the optimum chain length and head group-determined the inventors will return to the question of the need for one or two substituents at the 3- and/or 3, 6- positions alluded to above and the possibility of different 3, 6-substituents. By way of example the inventors set out a preparation of 3-chloro 6-methyoxycarbazole 21, which takes advantage of the recent advances in palladium catalyzed acrylation of amines and annilines described by the Hartwig and Buchwald groups (Hartwig et al., 1999; Wolfe et al., 1998, 1999; Wolfe and Buchwald, 1999). This is then followed by a second palladium mediated reaction, namely the oxidation cyclization of the diarylymine to the carbazole, which is one of the best known methods for the formation of this nucleus (Akermark et al., 1975). This palladium-mediated coupling and oxidative cyclization chemistry is very versatile and general and will enable use to prepare most differentially 3-6-disubstituted carbazoles that the inventors might require.

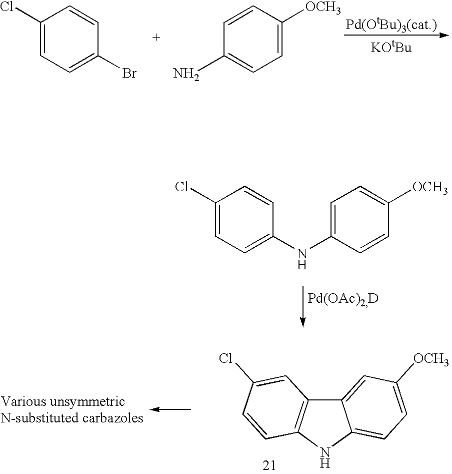

TABLE 1
Examples of Specific Carbazole Compounds

| ID | Structure | ID | Structure |
|---|---|---|---|
| INF801- 1649 | 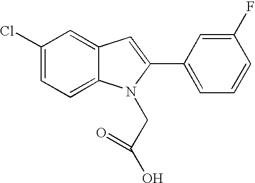 | S243213 | 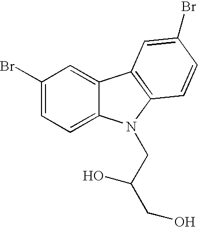 |
| INF801-749 | 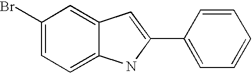 | INF801 | 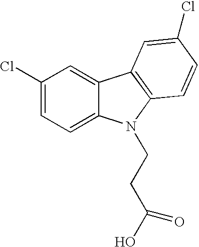 |
| INF801-822 | 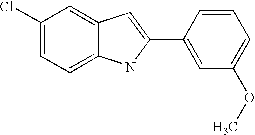 | INF801-1563 | 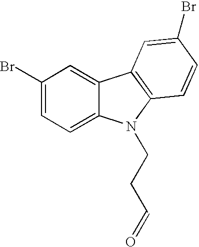 |
| INF801-834 | 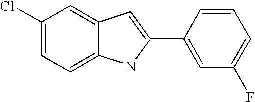 | INF801-1564 | 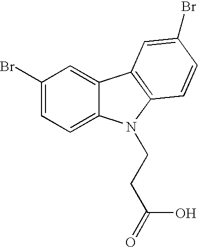 |
| DV 236683 | 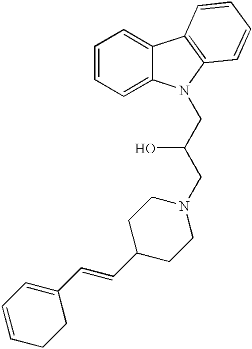 | DV 236687 | 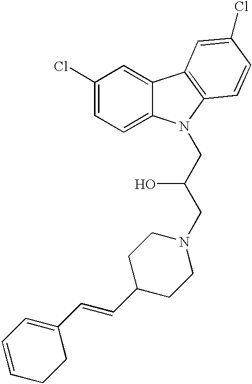 |
| DV 236818 | 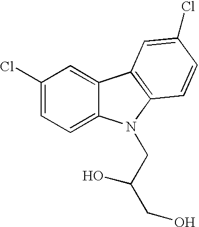 | INF801-1558 | 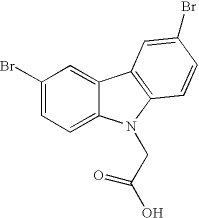 |
| INF801- 1574 | 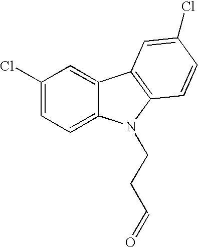 | INF801-1569 | 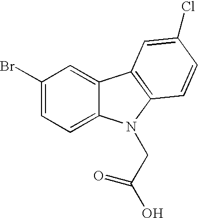 |
| DV 236817 | 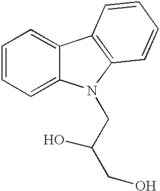 | INF801-1640 | 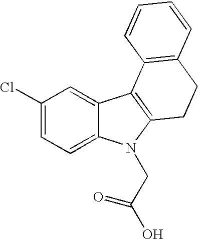 |
| INF801- 1589-2 | 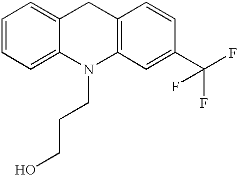 | INF801-1629 | 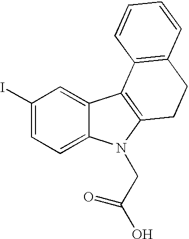 |
| INF801- 1642-2 | 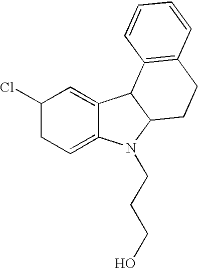 | INF801-737 | 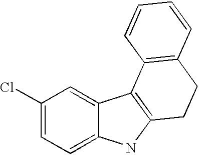 |
| INF801-731 | 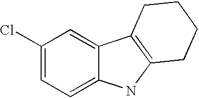 | | |

Analogs of INF 802 (Triptycenes): Initially fourteen commercially available triptycene compounds that were tested and found to fall into two broad categories: those carrying carbon substituents at the bridgehead (or 9-) position, which are active, and those unsubstituted at the bridgehead but substituted around the periphery of the molecule and which are inactive. One 9-substituted triptycene (S106615) was found to be inactive. It was apparent that molecules bearing a polar organic head group on the 9- position are active provided that this group is not branched close to the nucleus of the molecule, as is the case with S106615.

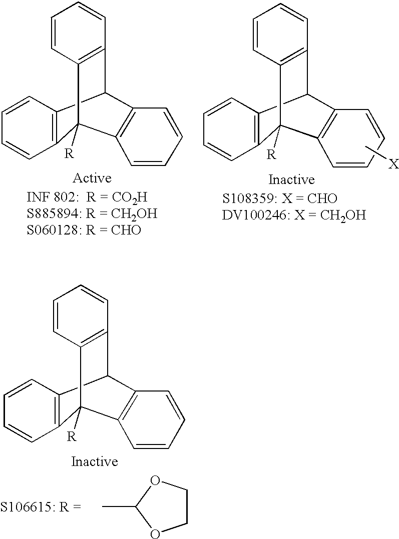

The inventors began by preparing a series of homologous saturated acids (22) with a view to probing the optimum length of the chain. Each of these acids was converted into the corresponding methyl ketones (23), esters (24) and reduced to the alcohols (25) by routine procedures. The alcohols were also esterified to give the acetates (26). Comparison of 22-26 within a particular chain length revealed the optimal head group and suggested other modifications to the functionality, while the chain length (n) was systematically extended until the optimum length was achieved. From this analysis the inventors found that extending the chain by 1 or 3, resulted in either a slight decrease in the activity, or equivalent activity, from the INF 802 compound. However, upon extending the chain length by either 2 or 4 carbons from that in INF 802, there is a 2 to 4 fold increase in the activity. Various functional (head) groups were tried, suggesting that polar groups like a carboxylic acid, a primary alcohol, or an aldehyde show the most activity, whereas esters, secondary alcohols, ketones, trifluoromethyl-ketones, and unsaturated chains linking the head groups to the triptycene showed a decrease in their activities.

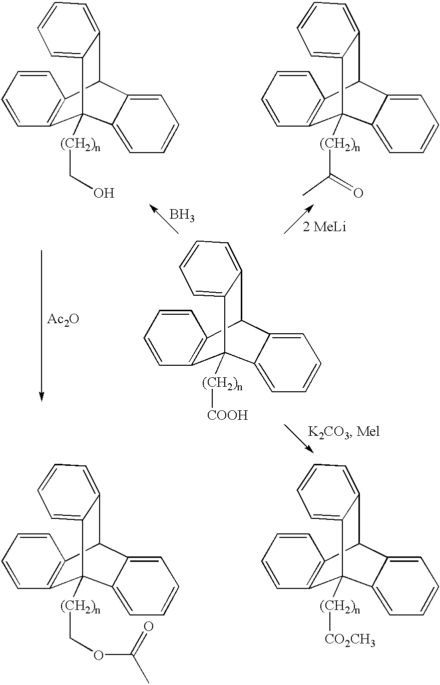

The chemistry of simple triptycene derivatives is both straightforward and well understood. By far, the most direct and efficient route is the cycloaddition of benzyne to substituted anthracenes as discussed in Organic Synthesis protocol (Wittig, 1963). The benzyne cycloaddition approach remains the optimum available method, as more recently demonstrated by Oki's extensive use in his synthesis of hindered triptycene derivatives (Oki et al., 1997), although prolysis of anthranilic acid has now been substituted for elimination from bromoflourobenzene as the most suitable means of generating the benzyne. The anthranilic acid/anthracene method was used by chemists at Lilly to generate a wide variety of 9-substituted triptycene derivatives, which were diversified by extensive functional group modifications, for an anti-inflammatory discovery program in the 1960's (Komfeld et al., 1965). Indeed, compounds 22 (n=2 and 7), 24 (n=2), 25 (n=2) were included in the Lilly study and the inventors did not anticipate any significant problems repeating this well-described work. Chains of different lengths were readily prepared by standard Wittig olefinations of the aldehyde S060128, using commercial carboxyalkylphosphonium salts, followed by hydrogenation of the alkene. Aldehyde S060128 was a member of the original library screened but may be obtained in sufficient quantities for use as starting material by cycloaddition of benzene with the diethylacetal of anthracene-9-carboxaldehyde as set out in the same Lilly study (Komfeld et al., 1965).

One area of space that had not been probed at all is the triptycene 10-position. Thus, having determined the optimum head group and chain length as set out above, the inventors proceeded to introduce a second lipophilic substituent at that site. 9, 10-Disubstituted triptycene derivatives are readily available from 9, 10-dichlorotriptycene (27), itself available directly by the cycloaddition route (Maerkl & Mayr, 1974) by metallation with BuLi, quenching with a first electrophile, a second metallation and a final quenching (Maerkl and Sejpka, 1985). By way of example the inventors set out below a synthesis of a 10-benzyl-9-methoxycarbonylethyletriptycene (29) using this type of chemistry. From this analysis it was found that a second substituent did not increase a compounds activity, but either gave near equivalent activity to the singly substituted triptycene or negated the activity all together. Due to the disappointing lack of increased activities in the di-substituted triptycenes, it was concluded that the singly substituted triptycenes were the best compounds to pursue.

A list of specifically contemplated triptycene derivatives is shown in Table 2.

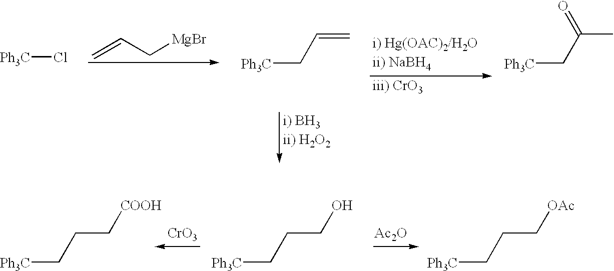

TABLE 2
Examples of Specific Triptycene Compounds

| ID | Structure | ID | Structure |
|---|---|---|---|
| INF802- 1403 | 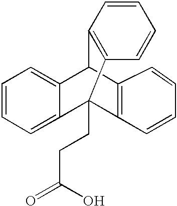 | INF802 | 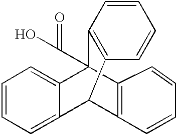 |
| INF802- 1405 | 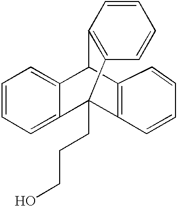 | S114421 | 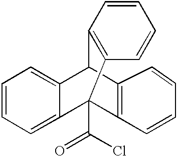 |
| INF802- 1412 | 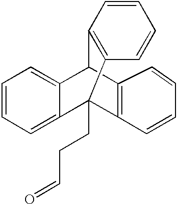 | INF802-1543 | 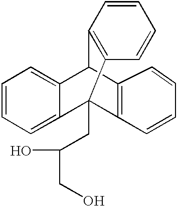 |
| INF802- 1417 | 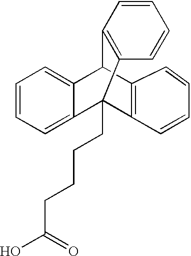 | INF802-1447 | 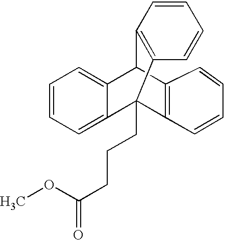 |
| INF802- 1419 | 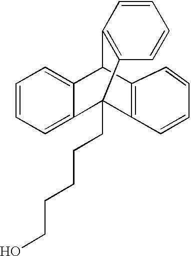 | INF802-1450 | 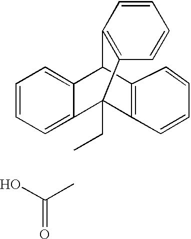 |
| INF802- 1421 | 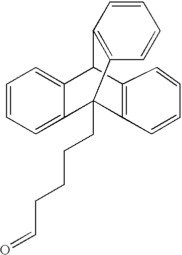 | INF802-1521 | 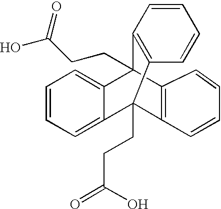 |
| INF802- 1432 | 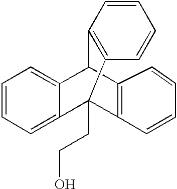 | INF802-1433 | 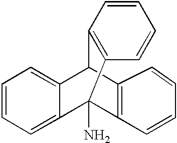 |
| INF802- 1444 | 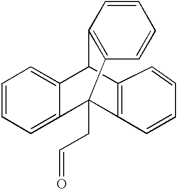 | S060128 | 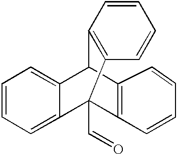 |
| INF802- 1446 | 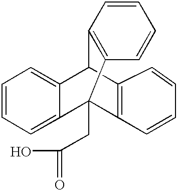 | INF802-1490 | 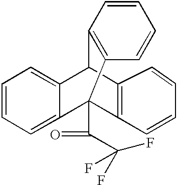 |

A further series of compounds that the inventors investigated were derived by replacing the triptycene moiety by the triphenylmethyl group. Such compounds were obtained inter alia by the action of Grignard reagents on triphenylmethyl chloride (Nesemeyanov and Perevalov, 1954). The scheme below gives the example of addition of alkylmagnesium bromide to triphenylmethyl chloride and illustrates how the ensuing alkene may be converted into any of the desired head groups. Higher and lower homologs are readily accessible by use of the appropriate Grignard reagent. A list of specifically contemplated compounds is shown in Table 3.

TABLE 3
Examples of Specific Triphenyl Compounds

| ID | Structure | ID | Structure |
|---|---|---|---|
| INF802- 1439 | 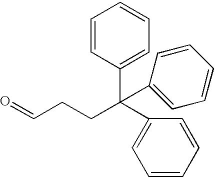 | 78, 120-5 | 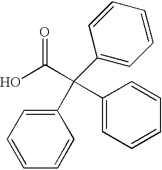 |
| INF802- 1440 | 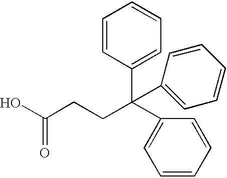 | INF802-1430-1 | 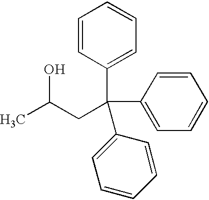 |

V. Additional Antifungal Treatments

In addition, the present invention also provides for new multi-drug therapy regimens. While many fungal infections may be effectively treated by a combination of an azole and a potentiating agent, comprising a carbazole analog or a triptycene, other infections may be treated more effectively using one or more additional agents. Such multi-drug combinations may reduce the amount of drug needed (and hence the side effects ensuing therefrom), may more quickly limit or eliminate the infection, and may more effectively treat, or even prevent, drug resistant fungi.

Traditional antifungal treatments that are suitable for use in combination with the present invention include polyenes, amphotericin B, filipin, nystatin, allylamines (terbinafine and naftifine), echinocandins (caspofungin or MK-0991, V-echinocandin, FK643), sordarins, azosordarins, flucytosine and griseofolvin. One or more of these agents may be combined with the azole+potentiator before, during or after that treatment.

VI. Pharmaceutical Formulations, Kits and Combination Therapies

Pharmaceutical Formulations: Pharmaceutical compositions of the present invention will generally comprise an effective amount of the azole potentiator dissolved or dispersed in a pharmaceutically acceptable carrier or aqueous medium. The pharmaceutical composition may further comprise an antifungal azole composition.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The azole potentiator of the present invention will often be formulated for parenteral administration, e.g., formulated for injection via the intravenous, intramuscular, sub-cutaneous or other such routes, including direct instillation into an infected or diseased site. The preparation of an aqueous composition that contains an azole potentiator agent as an active ingredient will be in known to those of skill in the art in light of the present disclosure. Typically, such compositions can be prepared as injectables, either as liquid solutions or suspensions; solid forms suitable for using to prepare solutions or suspensions upon the addition of a liquid prior to injection also can be prepared; and the preparations also can be emulsified.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The azole potentiator compositions can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. Formulations are easily administered in a variety of dosage forms, such as the type of injectable solutions described above, but drug release capsules and the like also can be employed.

Suitable pharmaceutical compositions in accordance with the invention will generally include an amount of the azole potentiator admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. It should be appreciated that, for human administration, preparations should meet sterility, pyrogenicity, general safety and purity standards as required by the FDA Office of Biological Standards.

The therapeutically effective doses are readily determinable using an animal model, as shown in the studies detailed herein. Experimental animals bearing bacterial or fungal infection are frequently used to optimize appropriate therapeutic doses prior to translating to a clinical ah environment. Such models are known to be very reliable in predicting effective anti-bacterial and antifungal strategies.

In addition to the compounds formulated for parenteral administration, such as intravenous or intramuscular injection, other pharmaceutically acceptable forms also are contemplated, e.g., tablets or other solids for oral administration, time release capsules, liposomal forms and the like. Other pharmaceutical formulations may also be used, dependent on the condition to be treated.

For oral administration, the azole potentiators of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

Kits: The present invention also provides therapeutic kits comprising the azole potentiators described herein. Such kits will generally contain, in suitable container means, a pharmaceutically acceptable formulation of at least one inhibitor in accordance with the invention. The kits may also contain other pharmaceutically acceptable formulations, such as those containing antibiotics such as azoles.

The kits may have a single container means that contains the azole potentiator, with or without any additional components, or they may have distinct container means for each desired agent. Certain preferred kits of the present invention include an azole potentiator, packaged in a kit for use in combination with the co-administration of an azole. In such kits, the azole potentiator and the azole may be pre-complexed, either in a molar equivalent combination, or with one component in excess of the other; or each of the azole potentiator and azole components of the kit may be maintained separately within distinct containers prior to administration to a patient. This is exemplary of the considerations that are applicable to the preparation of all such azole potentiator kits and kit combinations in general.

When the components of the kit are provided in one or more liquid solutions, the liquid solution is an aqueous solution, with a sterile aqueous solution being particularly preferred. as However, the components of the kit may be provided as dried powder(s). When reagents or components are provided as a dry powder, the powder can be reconstituted by the addition of a suitable solvent. It is envisioned that the solvent may also be provided in another container means.

The container means of the kit will generally include at least one vial, test tube, flask, bottle, syringe or other container means, into which the azole potentiator, and any other desired agent, may be placed and, preferably, suitably aliquoted. Where additional components are included, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the azole potentiator to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

Combination therapy: Fungal intrinsic and acquired resistance to antibiotics represents a major problem in the clinical management of fungal infections. One way of overcoming this hurdle is to combine traditional antifungal azoles with agents that enhance the antifungal activity of the azole. Such a combination therapy would be conceptually similar to the already widely used combinations of β-lactam or cephalosporin antibiotics with inhibitors of β-lactamase. In fact, one such combination, augmentin, has become the most frequently prescribed antibiotic preparation in the United States. The inventors propose that the clinical use of azoles in combination with a potentiator of azole activity that confers fungicidal activity should dramatically improve the clinical efficacy of current antifungal agents, especially in immunocompromised individuals.

To kill fungi, inhibit fungal cell growth, or otherwise reverse or reduce the suppressing effect on the emergence of drug-resistant variants using the methods and compositions of the present invention, one would generally contact a “target” cell with an azole and an potentiator of azole activity, as discussed herein. The compositions would be provided in a combined amount effective to kill fungi or inhibit fungal cell growth. This process may involve contacting the cells with the azole and the potentiator(s), and optionally other antifungal factor(s) at the same time. This may be achieved by contacting the cell with a single composition or pharmacological formulation that includes both agents, or by contacting the cell with two distinct compositions or formulations, at the same time, wherein one composition includes the azole and the other includes the potentiator.

The azole treatment may precede or follow the azole potentiator by intervals ranging from minutes to hours to days. In embodiments where the potentiator and azole are administered separately, one would generally ensure that a significant period of time did not expire between the time of each delivery, such that the azole and potentiator would still be able to exert an advantageously combined effect on abrogating the fungal infection. In such instances, it is contemplated that one would administer both modalities within about 12-24 hours of each other and, more preferably, within about 6-12 hours of each other. It may be that in order to sensitize the fugal cells to the azole treatment, the potentiator is administered for a sufficient period of time (1, 2 3, 4, 5, 6, 7, 8, 12, or 24 hours) prior to the azole treatment. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Equally it may be necessary to administer multiple doses of the potentiator in order to sensitize the bacterial cells to the azole treatment.

It also is conceivable that more than one administration of either azole or the potentiator will be desired. Various combinations may be employed, where the potentiator is “A” and the azole is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve fungal cell killing, both agents are delivered to a cell in a combined amount effective to kill the cell and remove the infection.

In certain embodiments, the antifungal azoles of the present invention may be used in combination with an enhancing agent to combat fungal infection. Such antifungal agents include but are not limited to chlormidazole, clotrimazole, miconazole, isoconazole, ketoconazole, econazole, bifonazole, butoconazole, democonazole, fenticonazole, lanoconazole, lombazole, oxiconazole, sertaconazole sulconazole, tioconazole, UR-9746, UR-9751 vibunazole, fluconazole, terconazole, genaconazole, itraconazole, voriconazole, posaconazole, ravuconazole, parconazole, SS 750, R120758, , R-102557, T-8581, TAK 456, TAK 457, BMS 207147 and SYN 2869.

The skilled artisan is directed to “the Physicians Desk Reference” 52nd Edition, in order to find detailed specific disclosure regarding particular azoles. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA Office of Biologics standards.

The inventors propose that the local or regional delivery of potentiator and/or the azole compositions to patients will be a very efficient method for delivering a therapeutically effective composition to counteract the clinical disease. Alternatively, systemic delivery of potentiator and/or the azole may be the most appropriate method of achieving therapeutic benefit from the compositions of the present invention.

VII. Screening for Antifungal Drug Combinations

Thus, in one aspect of the present invention, there is provided a method of screening for potentiators of azole antifungal activity. In its most basic form, the method comprises:

(a) providing a fungal cell;

(b) contacting said cell with an azole and a candidate potentiator substance; and

(c) comparing the antifungal activity of said azole with the antifungal activity of said azole in the absence of said candidate potentiator substance.

The method may further comprise comparing the antifungal activity of the candidate potentiator substance in the absence of the azole. Suitable fungi include, but are not limited to Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus Candida albicans is particularly contemplated. The fungus may also be resistant to azole treatment.

Suitable antifungal agents include the azoles chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 and SYN 2869. The assay can be further modified to determine conversion of the azole from fungistatic to fungicidal.

As the analogs of the lead compounds are synthesized, their inhibitory activities against Candida albicans will be further characterized. In a first series of tests, the inventors may determine the concentration of each analog required to inhibit post-MIC growth of C. albicans (strain ATCC 90028). This can be done by titrating each analog in a medium containing fluconazole at a concentration higher than the MIC in 96 well plates. Analogs alone will be tested as a control to evaluate their fungitoxicity.

Checkerboard microdilution experiments may be performed with each active analog and fluconazole. The minimal fungicidal concentration will be determined by the presence of different concentrations of fluconazole by plating out cells in wells with no visible growth and comparing CFUs to the original inoculum. For promising analogs, those with lower minimal active concentration than that of the lead compound, the inventors will perform time-kill tests to assess their fungicidal effect with fluconazole. This will help verify that their ability to inhibit post-MIC growth remains associated with their killing effect on the azole-treated cells. The experiments also will be performed with serum-supplemented medium to verify that analogs are not binding to serum proteins and thus lose activity.

Each promising analog also may be tested for its toxicity to mammalian cells. The IC₅₀ of each analog for HeLa cells after a 72-hour incubation will be determined in an MTS assay. Finally, for each analog, the inventors will determine solubility properties. Obviously, compounds whose minimal active concentration is close to the limit of their solubility are not of practical value. Therefore, the inventors will try to develop compounds whose active concentrations are far lower than their maximal concentration in solution and the IC₅₀ in the toxicity experiments.

The results of these tests were subjected to a quantitative structure activity relationship analysis to guide us in further synthetic efforts. The two lead compounds, INF 801 and INF 802, a carbazole and a triptycene, respectively, belong to structurally very rigid systems. Because of this, they were ideal for performing a 3D-QSAR/CoMFA (Cramer et al., 1988). Upon completion of the synthesis and testing of more derivatives of these two compounds, a 3D-QSAR/CoMFA analysis was performed. The CoMFA analysis (Zhou et al, 2000), performed with Sybyl 6.7 (Tripos, Inc.) was achieved by aligning common pharmacaphore points (aromatic rings for both types of compounds) within the set of compounds using DISCO (Martin et al., 1993). The CoMFA analyzed the steric and electrostatic components of these two sets of compounds. However, due to the small number of substituents on these base structures, along with the fact that only a few types of functional groups showed activity, the CoMFA results did not suggest any new areas of interest. The triptycene CoMFA results only suggested keeping the bulky groups toward the center of the molecule and the negatively charged regions some distance away. For the carbazoles, a reliable CoMFA model could not be found. This could be due to the fact that the carbazoles did not have a wide enough range in the activities, despite having a variety of substituents. However, it is clear, as was in the triptycenes, any polar, negatively charged group(s) are favored to be some distance from the center carbazole structure. Considering all the CoMFA, synthetic, and activity results, it was decided to alter the base ring structure, trying triphenylmethyl analogs, substituted indoles, in addition to inclusion of another heteroatom in the center ring of the carbazole, opposite the nitrogen.

VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Screening of a Chemical Library for Potentiators of Fluconazole Activity Against Candida albicans

The inventors screened a library of synthetic chemicals (Diverset™, ChemBridge Corp.), consisting of 9,600 “drug-like” compounds for activity in combination with the antifungal agent fluconazole. Three independent screens were performed. In one screen, the inventors looked for compounds that could potentiate the activity of fluconazole against C. glabrata, a Candida species intrinsically resistant to the drug. In another screen, the inventors looked for compounds that reversed the fluconazole resistant phenotype of the clinical isolate #17 of C. albicans, described by White, 1997. The resistance of this isolate is due to the cumulative effect of over expression of genes ERG11, MDRI and CDR1. The compounds in both screens were added at 10 μg/ml to wells of 96-well plates containing fluconazole at 16 μg/ml, approximately ¼-½ of the MICs of the tested strains. Unfortunately, nether screen yielded a promising lead compound significantly potentiating the activity of fluconazole against tester strains.

Far better results have been obtained with the third screen, in which the inventors were identifying compounds inhibiting post-MIC growth of wild-type C. albicans. Since fluconazole is only moderately fungistatic, C. albicans cells can grow, although at a lower rate, in the presence of high concentrations of fluconazole which far exceed the MIC₈₀ of the drug. The library was screened for compounds which, at concentration of 10 μg/ml, prevented visible growth of C. albicans cells (ATCC 36801) in YPD medium, when combined with a very high concentration (128 μg/ml) of fluconazole. The initial screen identified 226 such compounds.

The potency of these compounds was further tested by a series of two-fold dilutions both in the presence and absence of fluconazole (32 μg/ml). Ten of the compounds were found to inhibit the growth of C. albicans by themselves. Of greater interest to the present inventors were 64 compounds which displayed anti-Candida activity only in the presence of fluconazole at the concentration of 2.5 μg/ml or less. All of these strong hits were later confirmed using another strain of C. albicans (ATCC 90028), which is considered the standard strain for testing anti-Candida activity of chemicals, in a standard MOPS-buffered RPMI 1640 medium following the protocol recommended by NCCLS (NCCLS, 1997).

### EXAMPLE 2

### Preliminary In Vitro Toxicity Testing

HeLa cells were placed in 96-well plates. Twenty-four hours later, compounds were added to the wells to the final concentration of 10 μg/ml. Growth inhibition was determined 72 hours later by the MTS assay (Cell Titer 96 Aqueous assay, Promega). Only 30% of the hits showed growth inhibition of different degrees.

Growing human cells requires 10% serum (FCII, Hyclone) and serum albumin has high affinity for a variety of chemicals. To exclude the possibility that binding to albumin may shield toxicity, the inventors retested nontoxic hits with C. albicans that was growing in the standard MOPS-buffered RPMI 1640 medium supplemented with 10% serum. Yeast cells tend to be more sensitive to fluconazole in media with serum, presumably because of iron deprivation (Minn et al., 1997). The MIC of fluconazole was reduced to 2- to 4-fold in the presence of serum but cells continued to grow at the concentrations of fluconazole far exceeding the MIC. All the tested compounds retained the ability to inhibit this post-MIC growth in the presence of 1serum, indicating that serum albumin or other serum components did not alter substantially their activity.

### EXAMPLE 3

### Characterization and Quantification of Potentiating Effects of the Identified Lead Compounds

Sixteen lead compounds were identified that belonged to a wide variety of chemical classes. Based on their activities, they were categorized into three groups, discussed below.

Compounds inhibiting post-MIC growth. The largest group, consisting of 13 compounds, strongly inhibited post-MIC growth but had no effect on yeast viability and did not act on azole-resistant isolates. The most potent of these compounds was INF 799 (FIG. 1), a crown ether, which was active at a concentration as low as 2 ng/ml. When added at this or higher concentrations to the cultures of C. albicans (ATCC 36801, 90028, and nine azole-sensitive clinical isolates) growing with 16 μg/ml of fluconazole, INF 799 inhibited growth by approximately six-fold. At the same time, without fluconazole present, it had no effect on the growth of C. albicans, even at a concentration as high as 80 μg/ml. The MIC₈₀ of fluconazole also was not affected by this compound or any other compound of this group either in the sensitive, or in azole-resistant isolates described by White, 1997. It appears, therefore, that INF 799 and other members of this group of lead compounds specifically inhibit growth of the ergosterol-depleted yeast. Three other lead compounds, described below, not only demonstrated this property but also displayed additional beneficial characteristics which made them more promising candidates for further development as azole potentiators.

Compound inhibiting post-MIC growth and reversing MDR1-mediated azole resistance. Compound INF 800 (FIG. 1), at the concentration of 1 μg/ml and higher, in addition to inhibiting post-MIC growth of C. albicans, reduced the MIC₈₀ of fluconazole in sensitive isolates by two-four fold, i.e., from 0.5-1.0 μg/ml to 0.125-0.25 μg/ml. In some of resistant isolates, its effect was even more pronounced. In particular, the MIC of the isolate #4 (White, 1997) was reduced by this compound from 8 to 0.25 μg/ml. This isolate has been thoroughly characterized and found to have only one genetic change associated with azole resistance—over expression of the MDR1 transporter gene (White, 1997). It appears, therefore, that INF 800 has a dual activity. Like the majority of the identified leads, it inhibits the growth of ergosterol-depleted cells, and, additionally, inhibits MDR1-mediated azole resistance. Its moderate effect of the MIC of sensitive isolates is likely due to the inhibition of the MDR1 transporter expressed at the normal level.

Compounds displaying fungicidal activity in combination with azoles. The activity of compounds INF 801 and 802 is best illustrated in FIG. 2. Here, paper disks soaked in solutions of fluconazole (1 mg/ml), INF 801, INF 802 (both at 2.5 mg/ml in DMSO; DMSO controls showed no effect on fungal growth), or the combinations of fluconazole with these compounds, were placed on the surface of YPD agar plates inoculated with C. albicans. After 24 hours of incubation, fluconazole produced a pronounced zone of growth inhibition, but later, the slowly growing ergosterol-depleted C. Albicans largely caught up with the rest of the plate. These zones of inhibition can still be noticed in FIG. 2. Compounds INF 801 and INF 802 produced narrow zones of inhibition, indicating that they are toxic for Candida cells only at high concentrations. In fact, in liquid culture, compound INF 801 inhibited growth of C. Albicans only at concentrations exceeding 30 μg/ml, whereas INF 802 did not affect the growth of the yeast even at 80 μg/ml, the limit of its solubility. In contrast to individual compounds, the combinations of fluconazole with INF 801 or INF 802 produced large zones of inhibition lacking any growth of C. Albicans (FIG. 2). Most remarkably, no growth of Candida cells was detected within these zones even after a week of incubation.

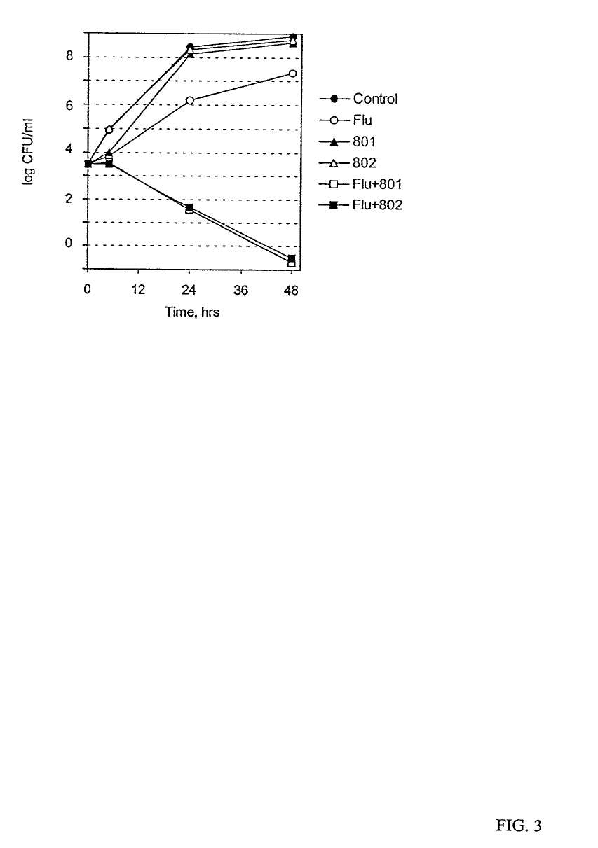

The explanation for this stable growth-inhibitory effect came from time-kill experiments, in which a liquid culture of C. Albicans was subjected to either fluconazole alone, to its combinations with INF 801 or ENF 802, or to these compounds alone. At different time points, the number of live cells in the cultures has been determined by plating appropriate dilutions on YPD plates and counting the number of colonies. FIG. 3, illustrating the results of this experiment, shows that fluconazole, even at a concentration as high as 32 μg/ml, merely slows down the growth of C. Albicans. After 48 hours, the number of CFUs (colony-forming units) in the fluconazole-containing culture increases by four orders of magnitude, as compared to five orders of magnitude in control. Compounds INF 801 and INF 802 by themselves have very little effect on the growth of the yeast, except that INF 801 reproducibly slows down their growth during the first several hours of incubation. Remarkably, combinations of only partially inhibitory fluconazole with the seemingly harmless compounds INF 801 or INF 802 cause massive death of the pathogen. The number of live cells drops by two orders of magnitude after 24 hours and by more than three orders of magnitude after 48 hours of incubation. In fact, plating of a milliliter of culture, which initially contained 3000 CFU/ml, yielded no colonies.

Testing Non-Albicans Candida Species. The growing number of Candida infections are caused by non-albicans species (Marichal et al., 1995; Pfaller et al., 1998). Many of these species, such as C. glabrota and especially C. kruzei, are inherently resistant to fluconazole, which is expressed in higher MIC values. The inventors tested the effects of INF 801 and INF 802 on the sensitivity of C. kruzei to fluconazole and found that these compounds have moderate reversing effect on the intrinsic resistance of this organism. The MIC of fluconazole was reduced 2-4 fold in their presence (Table 4). Considering that the MIC of fluconazole for C. kruzei is in the range of 8-128 μg/ml, this modest effect is unlikely to have significant chemical impact.

Importantly, however, both compounds were found to be strongly fungicidal in combination with fluconazole at the concentration exceeding its MIC. Fluconazole itself is strongly fungistatic for C. kruzei; unlike the situation with C. Albicans , the number of live cells does not increase substantially in the presence of 128 μg/ml of the drug. Neither does it decrease, indicating that ergosterol depletion in this species prevents growth but is not fungicidal. Addition of either INF 801 or INF 802, which are otherwise harmless to C. kruzei (not shown), causes massive killing of this yeast (Table 1). These results strongly suggest that both compounds can potentially enhance the effects of the existing azole drugs effective against C. kruzei, e.g., itraconazole (Nenoff et al., 1999), or the more potent azole drugs that may be developed in the future. Indeed, the fungicidal effect of INF 801 and INF 802 on C. Albicans was observed not only with fluconazole but also with other azoles, namely ketoconazole and itraconazole (see Tables 5 and 6), thus indicating that these potentiators, or their derivatives, can be combined with a variety of azole drugs.

TABLE 4
Response of Candida krusei* to the Lead Compounds

| | | Fluconazole + | Fluconazole + |
|---|---|---|---|
| | Fluconazole alone | INF 801 | INF 802 |
| MIC of fluconazole | 64 | 16 | 32 |
| (μg/ml) | | | |
| CFU/ml after 24 | 6230 | 20 | 20 |
| hours incubation** | | | |

*Strain M13C4
**Cells inoculated at 6000 CFU/ml were incubated with fluconazole (128 μg/ml) or its combinations with INF 801 and INF 802 (both at 20 μg/ml).

In summary, the performed screens of the chemical library identified a number of interesting potentiators of azole activity. Of these potentiators the inventors chose for further development two lead compounds, INF 801 and INF 802. These compounds demonstrate remarkable ability to kill yeast cells when combined with fungistatic, or even partially fungistatic concentrations of azole drugs exceeding MIC. Unlike compound INF 799, which is active at very low concentration (2 ng/ml, see above), INF 801 and INF 802 need to be present at relatively high concentrations to be active. When combined with high concentrations of fluconazole (16 μg/ml and more), their fungicidal activity is observed at 1.25 or 2.5 μg/ml, respectively. At concentrations of the azole that are clinically relevant (4 μg/ml), they are active only at ˜5-10 μg/ml.

### EXAMPLE 4

### Preliminary SAR Analysis with Commercially Available Analogs of Identified Lead Compounds

Eleven structural analogs of INF 801 and fourteen analogs of INF 802 were purchased from ChemBridge, Maybridge and Aldrich and tested for activity. Only some of these analogs displayed activity comparable to that of the lead compounds. Tables 5 and 6 demonstrate activities of each of the lead compounds and two of their most potent analogs. The numbers presented in these two tables indicate the -fold reduction in the number of live cells after 24 hour incubation with the combinations of azoles and potentiators. The higher the number, the higher the fungicidal activity of the combination. Note that concentrations of drags are somewhat different in the two tables, so the comparison between the two tables is not meaningful.

TABLE 5
Activity of INF 801 and Two of its Analogs Combined
with Azole Drugs Against C. albicans.

| Compound at | + Fluconazole, | + Ketaconazole, | + Itraconazole, |
|---|---|---|---|
| 10 μg/ml | 16 μg/ml | 0.5 μg/ml | 0.5 μg/ml |
| INF 801 | 48* | 28 | 30 |
| S243213 | 6.9 | 7.8 | 6.3 |
| DV 100735 | 27 | 152 | 109 |

*C. albicans strain ATCC 90028 was inoculated at 7600 CFU/ml with indicated combinations of drugs. The concentration of CFU's were determined after 24 hr. incubation. Presented numbers indicate fold reduction of the concentration of live cells as compared to the inoculum. See FIG. 1. for chemical structures.

TABLE 6
Activity of INF 802 and Two of its Analogs Combined
with Azole Drugs Against C. albicans.

| Compound at 5 | + Fluconazole, | + Ketaconazole, | + Itraconazole, |
|---|---|---|---|
| μg/ml | 16 μg/ml | 1 μg/ml | 1 μg/ml |
| INF 802 | 5.5 | 6.0 | 5.0 |
| S 885894 | 6.8 | 6.2 | 8.7 |
| S 060128 | 36 | 380 | 69 |

*C. albicans strain ATCC 90028 was inoculated at 8000 CFU/ml with indicated combinations of drugs. The concentration of CFU's was determined after 24 hr. incubation. Presented numbers indicate fold reduction of the concentration of live cells as compared to the inoculum. See FIG. 1 for chemical structures.

### EXAMPLE 5

### Biochemical Studies

The inventors conducted experiments to analyze the sterol composition of C. Albicans treated with different concentrations of fluconazole in the presence and absence of the lead compound INF 801. The isolation and TLC analysis of sterols was performed as described previously (Shimokawa & Nakayama, 1999). Since the presence of drugs lead to various o degrees of growth inhibition and, in the presence of INF 801, death of cells, the isolated sterols were normalized for the optical density of the yeast culture.

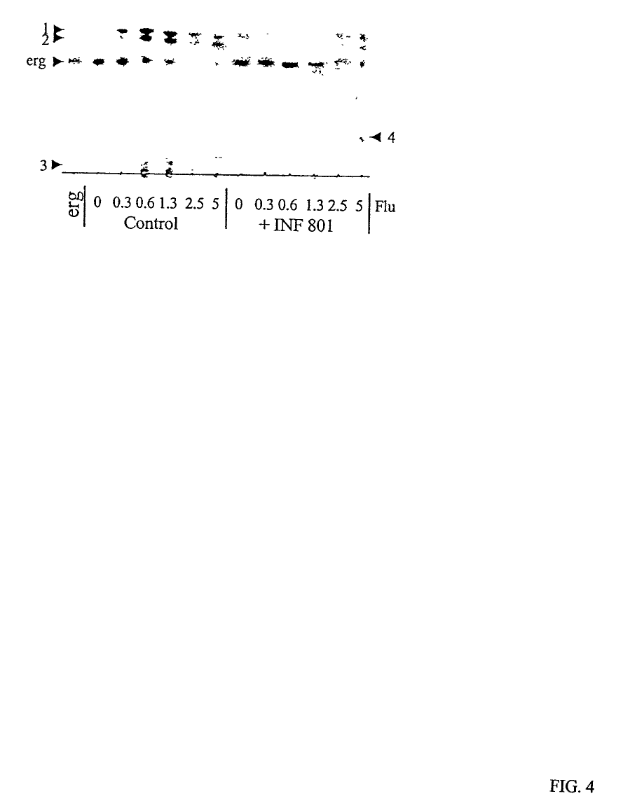

As FIG. 4 demonstrates, treatment of C. Albicans with increasing concentrations of fluconazole leads to a decrease in the amount of ergosterol. Instead, cells accumulate three sterol products marked in FIG. 4 as products 1, 2 and 3. The accumulation of products 1 and 2 has been reported previously by Shimokawa & Nakayama (1999), who identified them as 4,14-methylated and 4,4′,14-methylated sterols, respectively. The identity of product 3 remains unclear. In the presence of INF 801, fluconazole-induced depletion of ergosterol also occurs, surprisingly at somewhat higher concentrations of fluconazole. Most remarkable, however, is that the substituting sterols accumulate in much smaller amounts than in the control. Product 3 could not be detected at all, whereas product 2 appeared in the cells only at high concentrations of fluconazole. At the same high concentrations of the drug, accumulation of product 4, undetectable in control cells, was observed.

One can argue that these differences in sterol pools are merely a reflection of cell death occurring in the presence of INF 801. This, however, is unlikely, not only because the inventors attempted to compensate for cell death by normalizing cultures for optical density, but because significant differences in sterol patterns are observed at the concentrations of drugs (e.g., 0.6 μg/ml of fluconazole) at which yeast cells do not die even in the presence of INF 801. These experiments strongly suggest that compound INF 801 somehow interferes with the process of sterol metabolism.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

In accordance with the present invention there may be employed conventional molecular biology, microbiology, and recombinant DNA techniques within the skill of the art. Such techniques are explained fully in the literature. See, e.g., “Molecular Cloning: A Laboratory Manual,” Second Edition, Sambrook et al (1989); “DNA Cloning: A Practical Approach,” Volumes I and II, D. N. Glover, ed. (1985); “Oligonucleotide Synthesis,” M. J. Gait, ed. (1984); “Nucleic Acid Hybridization,” B. D. Hames & S. J. Higgins, eds. (1985); “Transcription And Translation,” B. D. Hames & S. J. Higgins, eds. (1984); “Animal Cell Culture,” R. I. Freshney, ed. (1986); “Immobilized Cells And Enzymes,” IRL Press, (1986); “A Practical Guide To Molecular Cloning,” B. Perbal, ed. (1984); “Molecular Biology of the Cell,” Alberts et al., ed. (1989); each of these references is specifically incorporated herein by reference.

IX. References

The following references, to the extent that they provide exemplary procedural or other details supplementary to those set forth herein, are specifically incorporated herein by reference.

Akermark etal., J. Org. Chem., 40:1365-1367, 1975.

Alexander and Perfect, Drugs, 54:657-678, 1997.

Blasi et al., Minerva Ginecol, 50:265-76, 1998.

Boerlin et al., J. Clin. Microbiol., 34:1235-1248, 1996.

Bohm et al., J. Med. Chem., 42: 458-477, 1999.

Borch, Org. Synth. Coll., 6:499-501, 1988.

Brajtburg and Bolard, Clin. Micro. Rev., 512-531, 1996.

Cameron et al., Antimicrob Agents Chemother, 37:2449-53, 1993.

Clancy et al., Int Arch Allergy Immunol, 118:349-50, 1999.

Cowen et al., J. Bacteriol, 182:1515-1522, 2000.

DeBrabander et al., Sabouraudia, 18:197-210, 1980.

Dixon et al., Public Health Report., 111:226-235, 1996.

Fong, Clin. Invest. Med., 17:426-31, 1994.

Fong et al., Genitourin Med., 69:44-6, 1993.

Fung-Tomc et al, Diagn Microbiol Infect Dis, 35:163-7, 1999.

Franz et al., Antimicrob. Agents Chemother., 42:3065-3072, 1998.

Franz et al., Mycoses, 42:453-458, 1999.

Gaber, Pol. J. Chem., 69:1301-1305, 1995.

Grotta et al., J. Org. Chem., 29:2474-2476, 1964.

Hartwig et al., J. Org Chem., 64:5575-5580, 1999.

Henderson and Hirvela, Arch. Urg., 131:330-337, 1996.

Iizawa et al., 40th Interscience Conference on Antimicrobial Agents and Chemotherapy, Abstract #1086, p. 199, 2000.

Irving et al., Sex Transm Infect, 74:334-8, 1998.

Johnson et al., Antimicrob Agents Chemother, 43:1260-3, 1999.

Kamai et al., 40th Interscience Conference on Antimicrobial Agents and Chemotherapy, Abstract #1080, p.200, 2000.

Kauffman and Carver, Drugs, 35:539-549, 1997.

Komfeld et al., J. Med. Chem., 8:342-347, 1965.

Kubota et al., Nippon Ishinkin Gakkai Zasshi, 39:213-8, 1998.

Lockhart et al., J. Clin. Microbiol., 34:767-77, 1996.

Lopez-Ribot et al., Antimicrob. Agents Chemother., 42:2932-2937, 1998.

Lynch et al., J Med. Vet. Mycol, 34:337-9, 1996.

Maerkl and Mayr, Tetrahedron Lett., 1817-1820, 1974.

Maerkl and Sejpka, Tetrahedron Lett., 26:5507-5510, 1985.

Marichal et al., Mycoses, 38:111-117, 1995.

Markham et al., Antimicrob. Agents Chemother., 43:2404-2408, 1999.

Marr et al., Antimicrob. Agents Chemother., 43:1383-1386, 1999.

Martin et al., J. Comp.-Aid. Mol. Des, 7:83-102, 1993.

Marchetti, O., et al., Antimicrob. Agents Chemother., 44: 2373-2381, 2000a.

Marchetti, O., et al., Antimicrob. Agents Chemother., 44: 2932-2938, 2000b.

Michalopoulos et al, Eur. J. Cardiothorac. Surg., 11:728-731, 1997.

Minn et al., Mycopathologia, 138:29-35, 1997.

Nenoff et al., Haustein UF Mycoses, 42:629-39, 1999.

Nesmeyanov and Perevalov, Akad. Nauk. SSSR, Ser. Khim., 1002-1006, 1954.

Oki et al., Bull. Chem. Soc. Jpn., 70:457-469, 1997.

Pacheco-Rios et al., Arch. Med. Res., 28:229-232, 1997.

Perlmutter, Conjugate addition reactions in organic synthesis, Oxford, 1992.

Pfaller et al., Diagn. Microbiol. Infect. Dis., 30:121-129, 1998.

Redding et al., Clin Infect Dis Feb., 18:240-2, 1994.

Revankar et al., J. Clin. Microbiol., 36:153-156, 1998a.

Revankar et al., Am J Med Jul., 105:7-11, 1998b.

Rex et al., Abstr. In Program and abstracts of the Infectious Diseases Society of America 34^{th} Annual Meeting Infectious Diseases Society of America, Washington, D.C., 74, p.49, 1996.

Sanglard, et al., Antimicrob. Agents and Chemother, 39:2378-2386, 1995.

Schroppel et al., J. Clin. Microbiol., 32:2646-2654, 1994.

Shigematsu et al, Jpn. J. Med. Mycol., 22, 195-201, 1981.

Shimokawa and Nakayama, Antimicrob. Agents Chemother., 36:1626-1629, 1992.

Shimokawa and Nakayama, Antimicrobial Agents and Chemother., 43:100-105, 1999.

Stepanova and Shiskina, Izv. Vyssh. Uchebn. Khim. Tekhnol, 10:298-300.

Sundberg, Esd., Pergamon, Oxford, 4:pp 313-376, 1984.

Tajima et al., Chem Pharm Bull, 48:694-707, 2000.

Takeda et al., 40th Interscience Conference on Antimicrobial Agents and Chemotherapy, Abstract #1080, p.198, 2000.

Tucker, J. Chem. Soc., 546-553, 1926.

Van Rheenan et al., Org. Syn. Coll., 6:342-348, 1988.

Vazquez et al., J. Infect. Dis., 170:1566-9, 1994.

White et al., Clinical Microbiology Reviews, 11:382-402, 1998.

White et al., Genitorin Med., 73:475-6, 1997.

White, Antimicrob Agents Chemother., 41:1482-7, 1997

Wittig et al., Triptycene. Org. Synth. Coll., 4:964-966, 1963.

Wolfe et al., Acc. Chem. Res., 31:805-818, 1998.

Wolfe and Buchwald, Angew. Chem. Int. Ed. Engl., 38:2413-2416, 1999.

Yotsuji et al., Antimicrob Agents Chemother, 41:30-4, 1997.

Zhou et al., J. Comp.-Aid. Mol. Des., 2000.

We claim:

1. A method for enhancing the antifungal action of azoles comprising contacting a fungal cell and an azole with a second agent comprising a triptycene, a triphenyl, a compound having the structure of formula (I′): 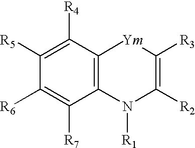
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4, or 5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R_{4,} R₅, R6, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons,
or a compound having the structure of formula (I″): 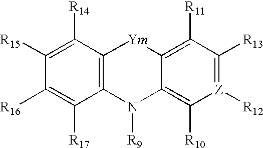
pharmaceutically acceptable salt or hydrate thereof, wherein:
Y is S or O;
m is 0 or 1;
Z is N or C;
R₉ is an —(CH₂)ₙCO₂R₁₈, —(CH₂)ₙCHO, —(CH₂)ₙOH, —(CH₂)ₙCHOHCH₃, (CH₂)ₙCHOHCH₂N(R₁₈)₂, —(CH₂)ₙ,CHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₁₈ is a H, or an alkyl chain up to 4 carbons;
n is 0, 1, 2, 3, 4,or5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₁₂, R₁₃, R₁₄, R,₁₅, R16, R₁₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons, with the proviso that when Z is N, R₁₂ is nonexistent,
or a compound having the structure of formula (I′″): 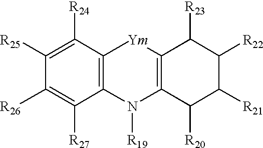
or a pharmaceutically acceptable salt or hydrate thereof, wherein:
Y is a S or O;
m is 0 or 1;
R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons;
X is a halogen;
R₂₈ is a H, or any alkyl chain up to 4 carbons;
n is 0, 1, 2, 3, 4,or5;
R₂₀, R₂₁ are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

2. The method of claim 1, wherein said second agent is an indole, a 1,4-dihydroquinolin, a pyridino[4,3-b]indole, a 5,6,7,8,9,10-hexahydroacridine, or a 5,10-dihydropydidino[3,4-b]quinoline.

3. The method of claim 1, wherein said second agent has the structure of formula (I′): 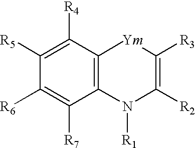
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons.

4. The method of claim 3, wherein:
m=0;
R₁ is a H or —CH₂CO₂H;
R₂ is a phenyl, 2-furanyl, m-fluorophenyl, or a pyridyl salt;
R₄, R₅, R₆ are a H, Cl, or Br; and
R₃, R₇ are a H.

5. The method of claim 3, wherein:
m=0;
R₂ is a phenyl, m-toluoyl, or m-fluorophenyl;
R₅ is a Cl or Br; and
R₁,R₃,R₄, R₆, R₇ are a H.

6. The method of claim 1, wherein said second agent has the structure of formula (I″): 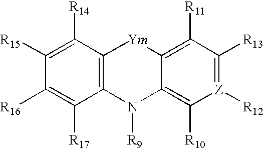
or a pharmaceutically acceptable salt or hydrate thereof, wherein:
Y is S or O;
m is 0 or 1;
Z is N or C;
R₉ is an —(CH₂)ₙCO₂R₁₈, —(CH₂)ₙCHO, —(CH₂)ₙOH, —(CH₂)ₙCHOHCH₃, (CH₂)ₙCHOHCH₂N(R₁₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₁₈ is a H, or an alkyl chain up to 4 carbons;
n is 0, 1, 2, 3, 4,or 5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino alkylamido, carbamyl alkyl ester, phenyl, or a branched or straight-chained alkyl group up to 4 carbons, with the proviso that when Z is N, R₁₂ is nonexistent.

7. The method of claim 6, wherein:
Y is a S;
m=0 or 1;
Z is a C;
R₉ is a 3-(4-styryl-piperazin-1-yl)propan-2-ol, —(CH₂)ₙOH, or —(CH₂)ₙCHOHCH₃,
n=1, 2, or3;
R₁₃, R₁₅ are a H, Cl, I, or Me; and
R₁₀, R₁₂, R₁₁, R₁₄, R₁₆, R₁₇ are a H.

8. The method of claim 6, wherein:
m=0;
Z is a C;
R₉ is a —(CH₂)ₙCO₂R₁₈, —(CH₂)ₙCHO, —(CH₂)ₙCHOHCH₂OH;
R₁₈ is a H;
n=1, 2, or 3;
R₁₃, R₁₅ is a Cl, Br, I, or H; and
R₁₀, R₁₂, R₁₁, R₁₄, R₁₆, R₁₇ are a H.

9. The method of claim 1, wherein said second agent has the structure of formula (I′″): 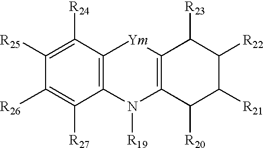
or a pharmaceutically acceptable salt or hydrate thereof, wherein:
Y is a S or O;
m is 0 or 1;
R₁₀ is an —(CH₂)ₙCO₂R₂, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons;
X is a halogen;
R₂₈ is a H, or any alkyl chain up to 4 carbons;
n is 0, 1, 2, 3, 4,or5;
R₂₀, R₂, are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

10. The method of claim 9, wherein:
m is 0;
R₁₉ is a H, —(CH₂)ₙCO₂H, or —(CH₂)ₙOH;
n is 1 or2;
R₂₂, R₂₃ are part of a benzene ring;
R₂₅ is a Cl, or I; and
R₂₀, R₂₁, R₂₄, R₂₆, R₂₇ are a H.

11. The method of claim 1, wherein said second agent is a carbazole.

12. The method of claim 6, wherein said second agent is a carbazole having the formula (I): 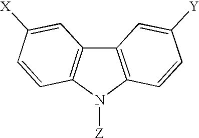
or a pharmaceutically acceptable salt or hydrate thereof, wherein:
X is an H, or a chloro, bromo, iodo, hydroxy, methoxy, amino, nitro, or dimethylamino;
Y is an H, or a chloro, bromo, iodo, hydroxy, methoxy, amino, nitro, or dimethylamino; and
Z is —(CH₂)ₙCO₂H, —CH(OH)CH₃, —(CH₂)ₙCHOHCH₂OH, —(CH₂)ₙCH₂OH, —(CH₂)ₙC(═O)(CH₂)ₘCH₃, or —(CH₂)ₙCO₂(CH₂)ₘCH₃, wherein n and m are independently 0, 1, 2, 3,
or Z is 4, 3-(4-styryl-piperazin-1-yl)propan-2-ol, an alkyl amide, or an alkoxyl.

13. The method of claim 12, wherein X and Y are halogen.

14. The method of claim 12, wherein X and Y are the same.

15. The method of claim 12, wherein X and Y are different.

16. The method of claim 12, wherein Z comprises 3 carbon atoms.

17. The method of claim 1, wherein said second agent is a triptycene having the formula (II′): 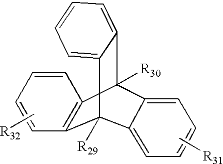
or a pharmaceutically acceptable salt or hydrate thereof, wherein:
R₂₉, R₃₀, R₃₁, R₃₂ are a H, —(CH₂)ₙCO₂R₃₃, —(CH₂)ₙOH, —(CH₂)ₙCHO, —(CH₂)ₙC(═O)CX₃, —(CH₂)ₙC(═O)Cl, —(CH₂)ₙC(═O)R₃₃, —(CH₂)ₙCHXR₃₃, —(CH₂)ₙCX₂R₃₃, —(CH₂)ₙN(R₃₃)₂, —(CH₂)ₙCH(OH)CH₃, —(CH₂)ₙCH(OH)CH₂OH, —CH═CHCH═CHCO₂R₃₃, —CH₂CH═CHCO₂R₃₃, —(CH₂)ₙCX₃, —C≡CCOR₄, —CON(R₃₃)₂, [1,3]-dioxolan-2-yl, phosphoryl dibenzylester, phosphoryl, sulfuryl, allyl, ethylene, nitro, or a halogen, wherein
X is a halogen;
R₃₃ is a H, or any alkyl chain up to 4 carbons; and
n=0, 1, 2, 3, 4, or 5.

18. The method of claim 17, wherein:
R₂₉ is a —(CH₂)ₙCO₂R₃₃, —(CH₂)ₙOH, or —(CH₂)ₙCHO, wherein
n=0, 2, 3, or 4;
R₃₃is H; and
R₃₀, R₃₁, R₃₂, are a H.

19. The method of claim 17, wherein said second agent is a triptycene having the formula (II): 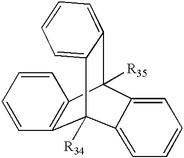
or a pharmaceutically acceptable salt or hydrate thereof, wherein:
R₃₄ is a —CO₂H, —CH₂OH, —CHO, —C(═O)Cl, —(CH₂)₃OCH₃, —(CH₂)ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; or branched hydroxyalkyl, branched alkoxyl, alkyl amide, or branched fluoro-, chloro-, bromo-, or iodo-alkyl;
R₃₅ is an H, or —CO₂H, —CH₂OH, —CHO, —C(═O)Cl, —(CH₂)₃OCH₃, —(CH₂)ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X⊚F, Cl, Br, or I, n=0, 1, 2, 3, 4, or 5, m=0, 1, 2, or 3, and p=0, 1, 2, or 3; or branched hydroxyalkyl, alkyl amide, branched alkoxyl, or branched fluoro-, chloro-, bromo-, or iodo-alkyl.

20. The method of claim 19, wherein R₃₄ and R₃₅ are different.

21. The method of claim 19, wherein R₃₅ is H.

22. The method of claim 19, wherein R₃₄ is a halogen.

23. The method of claim 19, wherein R₃₄ comprises two carbons.

24. The method of claim 1, wherein said fungal cell is selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus.

25. The method of claim 24, wherein said fungus is Candida.

26. The method of claim 25, wherein said Candida is Candida albicans.

27. The method of claim 1, wherein said fungus is resistant to azole treatment.

28. The method of claim 1, wherein said azole is chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

29. The method of claim 1, further comprising contacting said fungal cell and said azole and at least two distinct second agents.

30. The method of claim 1, wherein said azole is fungistatic in the absence of said second agent, and fungicidal in the presence of said second agent.

31. The method of claim 1, wherein said second agent is a triphenyl having the formula (III): 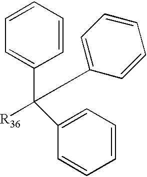
or a pharmaceutically acceptable salt or hydrate thereof, wherein:
R₃₆ is a H, —(CH₂)ₙCO₂R₃₇, —(CH₂)ₙOH, —(CH₂)ₙCHO, —(CH₂)ₙC(═O)CX₃, —(CH₂)ₙC(═O)Cl, —(CH₂)ₙN(R₃₇)₂, —(CH₂)ₙCH(OH)CH₃, —(CH₂)ₙCH(OH)CH₂OH, —CH═CHCH═CHCO₂R₃₇, —CH₂CH═CHCO₂R₃₇, —(CH₂)ₙCX₃, —C≡CCOR₃₇, —CON(R₃₇)₂, [1,3]-dioxolan-2-yl, phosphoryl dibenzylester, phosphoryl, sulfuryl, allyl, ethylene, nitro, or a halo; wherein
X is a halogen;
R₃₇ is a H, or any alkyl chain up to 4 carbons; and
n=0, 1, 2, 3, 4,or5.

32. The method of claim 31, wherein:
R₃₆ is a —(CH₂)ₙCO₂H or —(CH₂)ₙCHO; and
n=2.

33. A method of treating a fungal infection in a subject comprising administering to said subject, in antifungal amounts, an azole and a second agent comprising a triptycene, a triphenyl, a compound having the structure of formula (I′): 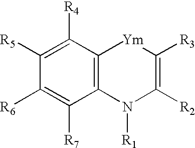
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin- 1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons,
or a compound having the structure of formula (I″): 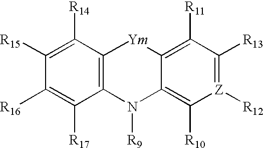
or a pharmaceutically acceptable salt or hydrate thereof, wherein:
Y is S or O;
m is 0 or 1;
Z is N or C;
R₉ is an —(CH₂)ₙCO₂R₁₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₁₈ is a H, or an alkyl chain up to 4 carbons;
n is 0, 1, 2, 3, 4,or5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons, with the proviso that when Z is N, R₁₂ is nonexistent,
or a compound having the structure of formula (I′″): 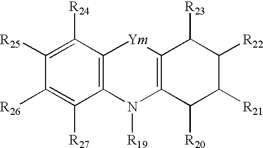
or a pharmaceutically acceptable salt or hydrate thereof, wherein:
Y is a S or O;
m is 0 or 1;
R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)_{CX}₃, —(CH₂)ₙCHOHCX₃, allyl, benzyl, H, or an alkyl chain up to 4 carbons;
X is a halogen;
R₂₈ is a H, or any alkyl chain up to 4 carbons;
n is 0, 1, 2, 3, 4, or 5;
R₂₀, R₂₁, are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

34. The method of claim 33, wherein said second agent is an indole, a 1,4-dihydroquinolin, a pyridino[4,3-b]indole, a 5,6,7,8,9,10-hexahydroacridine, or a 5,10-dihydropydidino[3,4-b]quinoline.

35. The method of claim 33, wherein said second agent has the structure of formula (I′): 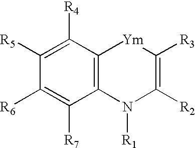
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons.

36. The method of claim 35, wherein:
m=0;
R₁ is a H or —CH₂CO₂H;
R₂ is a phenyl, 2-furanyl, m-fluorophenyl, or a pyridyl salt;
R₄, R₅, R₆ are a H, Cl, or Br; and
R₃, R₇ are a H.

37. The method of claim 35, wherein:
m=0;
R₂ is a phenyl, m-toluoyl, or m-fluorophenyl;
R₅ is a Cl or Br; and
R₁, R₃, R₄, R₆, R₇ are a H.

38. The method of claim 33, wherein said second agent has the structure of formula (I″): 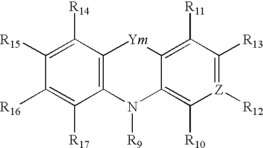
or a pharmaceutically acceptable salt or hydrate thereof, wherein:
Y is S or O;
m is 0 or 1;
Z is N or C;
R₉ is an —(CH₂)ₙCO₂R₈, —(CH₂)ₙCHO, —(CH₂)ₙOH, —(CH₂)ₙCHOHCH₃, (CH₂)ₙCHOHCH₂N(R₁₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₁₈ is a H, or an alkyl chain up to 4 carbons;
n is 0, 1, 2, 3, 4,or5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino alkylamido, carbamyl alkyl ester, phenyl, or a branched or straight-chained alkyl group up to 4 carbons, with the proviso that when Z is N, R₁₂ is nonexistent.

39. The method of claim 38, wherein:
Y is a S;
m=0 or 1;
Z is a C;
R₉ is a 3-(4-styryl-piperazin-1-yl)propan-2-ol, —(CH₂)ₙOH, or —(CH₂)ₙCHOHCH₃;
n=1, 2, or 3;
R₁₃ R₁₅ are a H, Cl, I, or Me; and
R₁₀, R₁₂, R₁₁, R₁₄, R₁₆, R₁₇ are a H.

40. The method of claim 38, wherein:
m=0;
R₉ is a —(CH₂)ₙCO₂R₈, —(CH₂)ₙCHO, —(CH₂)ₙCHOHCH₂OH;
R₁₈ is a H;
n=1, 2, or 3;
Z is a C;
R₁₃, R₁₅ is a Cl, Br, I, or H; and
R₁₀, R₁₂, R₁₁, R₁₄, R₁₆, R₁₇ are a H.

41. The method of claim 33, wherein said second agent has the structure of formula (I′″): 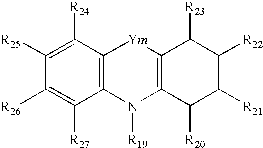
or a pharmaceutically acceptable salt or hydrate thereof, wherein:
Y is a S or O;
m is 0 or 1;
R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons;
X is a halogen;
R₂₈ is a H, or any alkyl chain up to 4 carbons;
n is 0, 1, 2, 3, 4,or5;
R₂₀, R₂₁, are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

42. The method of claim 41, wherein:
m is 0;
R₁₉ is a H, —(CH₂)ₙCO₂H, or —(CH₂)ₙOH;
n is 1 or2;
R₂₂, R₂₃ are part of a benzene ring;
R₂₅ is a Cl, or I; and
R₂₀, R₂₁, R₂₄, R₂₆, R₂₇ are a H.

43. The method of claim 33, wherein said second agent is a carbazole.

44. The method of claim 38, wherein said second agent is a carbazole having the formula (I): 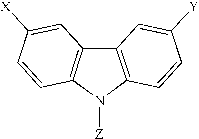
or a pharmaceutically acceptable salt or hydrate thereof, wherein:
X is an H, or a chloro, bromo, iodo, hydroxy, methoxy, amino, nitro, or dimethylamino;
Y is an H, or a chloro, bromo, iodo, hydroxy, methoxy, amino, nitro, or dimethylamino;
and
Z is —(CH₂)ₙCO₂H, —CH(OH)CH₃, —(CH₂)ₙCHOHCH₂OH, —(CH₂)ₙCH₂OH, —(CH₂)ₙC(═O)(CH₂)ₘCH₃, or —(CH₂)ₙCO₂(CH₂)ₘCH₃, wherein n and m are independently 0, 1, 2, 3,
or Z is 4, 3-(4-styryl-piperazin-1-yl)propan-2-ol, an alkyl amide, or an alkoxyl.

45. The method of claim 44, wherein X and Y are halogen.

46. The method of claim 44, wherein X and Y are the same.

47. The method of claim 44, wherein X and Y are different.

48. The method of claim 44, wherein Z comprises 3 carbon atoms.

49. The method of claim 33, wherein said second agent is a triptycene having the formula (II′): 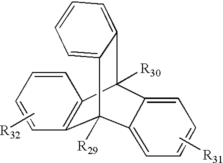
or a pharmaceutically acceptable salt or hydrate thereof, wherein:
R₂₉, R₃₀, R₃₁, R₃₂ are a H, —(CH₂)ₙCO₂R₃₃, —(CH₂),OH, —(CH₂)₂)ₙCHO, —(CH₂)ₙC(═O)CX₃, —(CH₂)ₙC(═O)Cl, —(CH₂)ₙC(═O)R₃₃, —(CH₂)ₙCHXR₃₃, —(CH₂)ₙCX₂R₃₃, —(CH₂)ₙN(R₃₃)₂, —(CH₂)ₙCH(OH)CH₃, —(CH₂)ₙCH(OH)CH₂OH, —CH═CHCH═CHCO₂R₃₃, —CH₂CH═CHCO₂R₃₃, —(CH₂)ₙCX₃, —CCOR₄, —CON(R₃₃)₂, [1,3 ]-dioxolan-2yl, phosphoryl dibenzylester, phosphoryl, sulfuryl, allyl, ethylene, nitro, or a halogen, wherein
X is a halogen;
R₃₃ is a H, or any alkyl chain up to 4 carbons; and
n=0, 1, 2, 3, 4, or5.

50. The method of claim 49, wherein:
R₂₉ is a —(CH₂)ₙCO₂R₃₃, —(CH₂)ₙOH, or —(CH₂)ₙCHO, wherein
n=0, 2, 3,or4;
R₃₃ is H; and
R₃₀, R₃₁, R₃₂, are a H.

51. The method of claim 49, wherein said second agent is a triptycene having the formula (II): 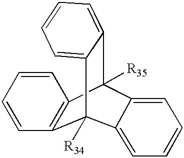
or a pharmaceutically acceptable salt or hydrate thereof, wherein:
R₃₄ is a —CO₂H, —CH₂OH, —CHO, —C(═O)Cl, —(CH₂)₃OCH₃, —(CH₂)ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; or branched hydroxyalkyl, branched alkoxyl, alkyl amide, or branched fluoro-, chloro-, bromo-, or iodo-alkyl; and
R₃₅ is an H, or —CO₂H, —CH₂OH, —CHO, —C(═O)Cl, —(CH₂)₃OCH₃, —(CH₂)ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; or branched hydroxyalkyl, alkyl amide, branched alkoxyl, or branched fluoro-, chloro-, bromo-, or iodo-alkyl.

52. The method of claim 51, wherein R₃₄ and R₃₅ are different.

53. The method of claim 51, wherein R₃₅ is H.

54. The method of claim 51, wherein R₃₄ is a halogen.

55. The method of claim 51, wherein R₃₄ comprises two carbons.

56. The method of claim 33, wherein said second agent is a triphenyl having the formula (III): 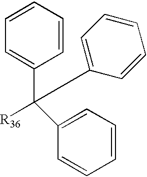
or a pharmaceutically acceptable salt or hydrate thereof, wherein:
R₃₆ is a H, —(CH₂)ₙCO₂R₃₇, —(CH₂)ₙOH, —(CH₂)ₙCHO, —(CH₂)ₙC(═O)CX₃, —(CH₂)ₙC(═O)Cl, —(CH₂)ₙN(R₃₇)₂, —(CH₂)ₙCH(OH)CH₃, —(CH₂)ₙCH(OH)CH₂OH, —CH═CHCH═CHCO₂R₃₇, —CH₂CH═CHCO₂R₃₇, —(CH₂)ₙCX₃, —C≡CCOR₃₇, —CON(R₃₇)₂, [1,3]-dioxolan-2-yl, phosphoryl dibenzylester, phosphoryl, sulfuryl, allyl, ethylene, nitro, or a halo; wherein
X is a halogen;
R₃₇ is a H, or any alkyl chain up to 4 carbons; and
n 0, 1, 2, 3, 4, or 5.

57. The method of claim 56, wherein:
R₃₆ is a —(CH₂)ₙCO₂H or —(CH₂)ₙCHO; and
n=2.

58. The method of claim 33, wherein said azole is chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-975 1, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

59. The method of claim 33, wherein said fungal infection is caused by a fungus is selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus.

60. The method of claim 59, wherein said fungus is Candida.

61. The method of claim 60, wherein said Candida is Candida albicans.

62. The method of claim 33, wherein said fungus is resistant to azole treatment.

63. The method of claim 33, wherein the azole and the second agent are delivered together.

64. The method of claim 33, wherein the azole and the second agent are delivered separately.

65. The method of claim 64, wherein the azole and the second agent are delivered within 2 hours of each other.

66. The method of claim 33, wherein one or both of the azole and the second agent are delivered intravenously.

67. The method of claim 33, wherein one or both of the azole and the second agent are administered repeatedly.

68. The method of claim 33, wherein the subject is an animal.

69. The method of claim 67, wherein the animal is a human.

70. The method of claim 33, further comprising administering at least two distinct second agents to said subject.

71. The method of claim 33, wherein said azole is fungistatic in the absence of said second agent, and fungicidal in the presence of said second agent.

72. A method of screening for potentiators of azole antifungal activity comprising:
(a) providing a fungal cell;
(b) contacting said cell with an azole and a candidate potentiator substance; and
(c) comparing the antifungal activity said azole with the antifungal activity of said azole in the absence of said candidate potentiator substance.

73. The method of claim 72, farther comprising comparing the antifungal activity of the candidate potentiator substance in the absence of said azole.

74. The method of claim 73, wherein said fungal cell is selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus.

75. The method of claim 74, wherein said fungus is Candida.

76. The method of claim 75, wherein said Candida is Candida albicans.

77. The method of claim 72, wherein said fungal cell is resistant to azole treatment.

78. The method of claim 72, wherein said azole is chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

79. The method of claim 72, wherein said azole is fungistatic in the absence of said second agent, and fungicidal in the presence of said second agent.

80. A pharmaceutical composition comprising an azole and a second agent comprising a triptycene, a triphenyl, a compound having the structure of formula (I′): 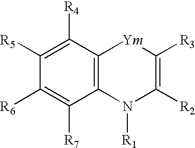
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons,
or a compound having the structure of formula (I″): 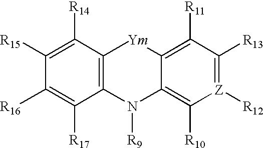
or a pharmaceutically acceptable salt or hydrate thereof, wherein:
Y is S or O;
m is 0 or 1;
Z is N or C;
R₉ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₂, R₃, R₄, R₆, R₇ are a hydrogen, a halogen, trifluoromethyl, thioalkyl, alkoxy, hydroxy, amino, alkylamino, dialkylamino alkylamido, carbamyl alkyl ester, phenyl, or a branched or straight-chained alkyl group up to 4 carbons, with the proviso that when Z is N, R₁₂ is nonexistent,
or a compound having the structure of formula (I′″): 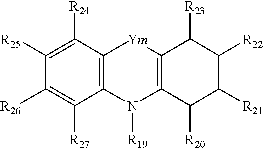
or a pharmaceutically acceptable salt or hydrate thereof, wherein:
Y is a S or O;
m is 0 or 1;
R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons;
X is a halogen;
R₂₈ is a H, or any alkyl chain up to 4 carbons;
n is 0, 1, 2, 3, 4,or5;
R₂₀, R₂₁ are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

81. The composition of claim 80, wherein said azole is chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-975 1, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

82. The composition of claim 80, wherein said second agent is an indole, a 1,4-dihydroquinolin, a pyridino[4,3-b]indole, a 5,6,7,8,9,10-hexahydroacridine, or a 5,10-dihydropydidino[3,4-b]quinoline.

83. The composition of claim 80, wherein said second agent has the structure of formula (I′): 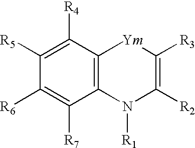
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons.

84. The composition of claim 83, wherein:
m=0;
R₁ is a H or —CH₂CO₂H;
R₂ is a phenyl, 2-furanyl, m-fluorophenyl, or a pyridyl salt;
R₄, R₅, R₆ are a H, Cl, or Br; and
R₃, R₇ are a H.

85. The composition of claim 83 wherein:
m=0;
R₂ is a phenyl, m-toluoyl, or m-fluorophenyl;
R₅ is a Cl or Br; and
R₁, R₃, R₄, R₆, R₇ are a H.

86. The composition of claim 80, wherein said second agent has the structure of formula (I″): 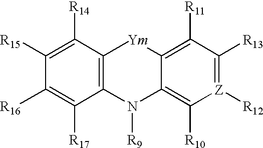
or a pharmaceutically acceptable salt or hydrate thereof, wherein:
Y is S or O;
m is 0 or 1;
Z is N or C;
R₉ is an —(CH₂)ₙCO₂R₈, —(CH₂)ₙCHO, —(CH₂)ₙOH, —(CH₂)ₙCHOHCH₃, (CH₂)ₙCHOHCH₂N(R₁₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino alkylamido, carbamyl alkyl ester, phenyl, or a branched or straight-chained alkyl group up to 4 carbons, with the proviso that when Z is N, R₁₂ is nonexistent.

87. The composition of claim 86, wherein:
Y is a S;
m=0 or 1;
R₉ is a 3-(4-styryl-piperazin-1-yl)propan-2-ol, —(CH₂)ₙOH, or —(CH₂)ₙCHOHCH₃;
n=1, 2, or 3;
R₁₃, R₁₅ are a H, Cl, I, or Me; and
R₁₀, R₁₂, R₁₁, R₁₄, R₁₆, R₁₇ are a H.

88. The composition of claim 87, wherein:
m=0;
R₉ is a —(CH₂)ₙCO₂R₁₈, —(CH₂)ₙCHO, —(CH₂)ₙCHOHCH₂OH;
R₁₈ is a H;
n=1, 2,or3;
R₁₃, R₁₅ is a Cl, Br, I, or H; and
R₁₀, R₁₁, R₁₃, R₁₄, R₁₆, R₁₇ are a H.

89. The composition of claim 80, wherein said second agent has the structure of formula (I′″): 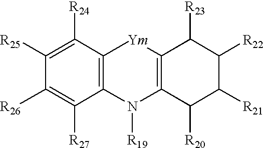
or a pharmaceutically acceptable salt or hydrate thereof, wherein:
Y is a S or O;
m is 0 or 1;
R₁₉ is an —(CH₂)ₙCO₂R₁₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons;
X is a halogen;
R₂₈ is a H, or any alkyl chain up to 4 carbons;
n is 0, 1, 2, 3, 4,or5;
R₂₀, R₂₁ are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

90. The composition of claim 89, wherein:
m is 0;
R₁₉ is a H, —(CH₂)ₙCO₂H, or —(CH₂)ₙOH;
n is 1 or2;
R₂₂, R₂₃ are part of a benzene ring;
R₂₅ is a Cl, or I; and
R₂₀, R₂₁, R₂₄, R₂₆, R₂₇ are a H.

91. The composition of claim 80, wherein said second agent is a carbazole.

92. The composition of claim 86, wherein said second agent is a carbazole having the formula (I): 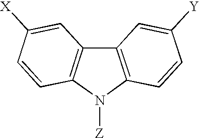
or a pharmaceutically acceptable salt or hydrate thereof, wherein:
X is an H, or a chloro, bromo, iodo, hydroxy, methoxy, amino, nitro, or dimethylamino;
Y is an H, or a chloro, bromo, iodo, hydroxy, methoxy, amino, nitro, or dimethylamino;
and
Z is —(CH₂)ₙCO₂H, —CH(OH)CH₃, —(CH₂)ₙCHOHCH₂OH, —(CH₂)ₙCH₂ OH, —(CH₂)ₙC(═O)(CH₂)ₘCH₃, or —(CH₂)ₙCO₂(CH₂)ₘCH₃, wherein n and m are independently 0, 1, 2, 3,
or Z is 4, 3-(4-styryl-piperazin-1-yl)propan-2-ol, an alkyl amide, or an alkoxyl.

93. The composition of claim 92, wherein X and Y are halogen.

94. The composition of claim 92, wherein X and Y are the same.

95. The composition of claim 92, wherein X and Y are different.

96. The composition of claim 92, wherein Z comprises 3 carbon atoms.

97. The composition of claim 80, wherein said second agent is a triptycene having the 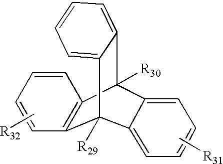
formula (II′):
or a pharmaceutically acceptable salt or hydrate thereof, wherein:
R₂₉, R₃₀, R₃₁, R₃₂ are a H, —(CH₂)ₙCO₂R₃₃, —(CH₂)ₙOH, —(CH₂)ₙCHO, —(CH₂)ₙC(═O)CX₃, —(CH₂)ₙC(═O)Cl, —(CH₂)ₙC(═O)R₃₃, —(CH₂)ₙCHXR₃₃, —(CH₂)ₙCX₂R₃₃, —(CH₂)ₙN(R₃₃)₂, —(CH₂)ₙCH(OH)CH₃, —(CH₂)ₙCH(OH)CH₂OH, —CH═CHCH═CHCO₂R₃₃, —CH₂CH═CHCO₂R₃₃, —(CH₂)ₙCX₃, —C═CCOR₄, —CON(R₃₃)₂, [1,3 ]-dioxolan-2-yl phosphoryl dibenzylester, phosphoryl, sulfuryl, allyl, ethylene, nitro, or a halogen, wherein
X is a halogen;
R₃₃ is a H, or any alkyl chain up to 4 carbons; and
n=0, 1, 2, 3, 4,or5.

98. The composition of claim 97, wherein:
R₂₉ is a —(CH₂)ₙCO₂R₃₃, —(CH₂)ₙOH, or —(CH₂)ₙCHO, wherein
n=0, 2, 3,or4;
R₃₃ is H; and
R₃₀, R₃₁, R₃₂, are a H.

99. The composition of claim 97, wherein said second agent is a triptycene having the formula (II): 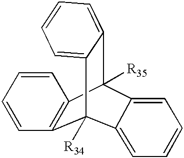
or a pharmaceutically acceptable salt or hydrate thereof, wherein:
R₃₄ is a —CO₂H, —CH₂OH, —CHO, —C(═O)Cl, —(CH₂)₃OCH₃, —(CH₂)ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; or branched hydroxyalkyl, branched alkoxyl, alkyl amide, or branched fluoro-, chloro-, bromo-, or iodo-alkyl; and
R₃₅ is an H, or —CO₂H, —CH₂OH, —CHO, —C(=O)Cl, —(CH₂)₃OCH₃, —(CH₂)ₙOH, —(CH₂)ₙC(═O)CH₃, —(CH₂)ₙCO₂Me, —(CH₂)ₙC(═O)(CH₂)ₘCH₃, —(CH₂)ₙCO₂(CH₂)ₘCH₃, —(CH₂)ₙCO₂H, —(CH₂)ₙOH, —(CH₂)ₙCXₚ, wherein X═F, Cl, Br, or I, n=0, 1, 2, 3, 4, or 5, m=0, 1, 2, or 3, and p=0, 1, 2, or 3; or branched hydroxyalkyl, alkyl amide, branched alkoxyl, or branched fluoro-, chloro-, bromo-, or iodo-alkyl.

100. The composition of claim 99, wherein R₃₄ and R₃₅ are different.

101. The composition of claim 99, wherein R₃₅ is H.

102. The composition of claim 99, wherein R₃₄ is a halogen.

103. The composition of claim 99, wherein R₃₄ comprises two carbons.

104. The composition of claim 80, wherein said second agent is a triphenyl having the formula (III): 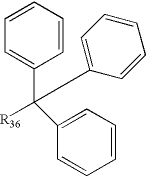
or a pharmaceutically acceptable salt or hydrate thereof, wherein:
R₃₆ is a H, —(CH₂)ₙCO₂R₃₇, —(CH₂)ₙOH, —(CH₂)ₙCHO, —(CH₂)ₙC(═O)CX₃, —(CH₂)ₙC(═O)Cl, —(CH₂)ₙN(R₃₇)₂, —(CH₂)ₙCH(OH)CH₃, —(CH₂)ₙCH(OH)CH₂OH, —CH═CHCH═CHCO₂R₃₇, —CH₂CH═CHCO₂R₃₇, —(CH₂)ₙCX₃, —C≡CCOR₃₇, —CON(R₃₇)₂, [1,3]-dioxolan-2-yl, phosphoryl dibenzylester, phosphoryl, sulfuryl, allyl, ethylene, nitro, or a halo; wherein
X is a halogen;
R₃₇ is a H, or any alkyl chain up to 4 carbons; and
n=0, 1, 2, 3, 4,or5.

105. The composition of claim 104, wherein:
R₃₆ is a —(CH₂)ₙCO₂H or —(CH₂)ₙCHO; and
n=2.

106. A method for suppressing the emergence of azole resistance in fungi comprising contacting a fungal cell during the course of azole therapy with a second agent comprising a triptycene, a triphenyl, a compound having the structure of formula (I′): 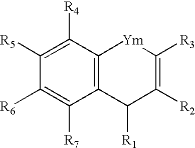
or a pharmaceutically acceptable salt or hydrate thereof, wherein:
Y is S or O;
m is 0 or 1;
R₁ is an —(CH₂)ₙCO₂R₈, —(CH₂)ₙCHO, —(CH₂)ₙOH, —(CH₂)ₙCHOHCH₃, (CH₂)ₙCHOHCH₂N(R₈)₂, —(CH₂)ₙCHOHCH₂OH, —(CH₂)ₙC(OH)₂CX₃, (—CH₂)ₙCHOHCX₃, 3-(4-styryl-piperazin-1-yl)propan-2-ol, allyl, benzyl, H, or an alkyl chain up to 4 carbons; wherein
X is a halogen;
R₈ is a H, or an alkyl chain up to 4 carbons;
n is 0, 1, 2, 3, 4,or5;
R₂ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₃ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₄, R₅, R₆, R₇ are a hydrogen, a halogen, trifluoromethyl, thioalkyl, alkoxy, hydroxy, amino, alkylamino, dialkylamino, alkylamido, carbamyl alkyl ester, phenyl, or a branched or straight-chained alkyl group up to 4 carbons,
or a compound having the structure of formula (I″): 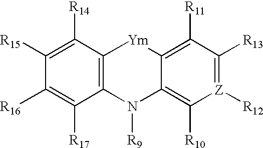
or a pharmaceutically acceptable salt or hydrate thereof, wherein:
Y is S or O;
m is 0 or 1;
Z is N or C;
R₉ is an —(CH₂)ₙCO₂R₁₈, —(CH₂)ₙCHO, —(CH₂)ₙOH, —(CH₂)ₙCHOHCH₃, (CH₂)ₙCHOHCH₂N(R₁₈)₂, —(CH₂)ₙCHOHCH₂OH, —(CH₂)ₙC(OH)₂CX₃, (—CH₂)ₙCHOHCX₃, 3-(4-styryl-piperazin- 1-yl)propan-2-ol, allyl, benzyl, H, or an alkyl chain up to 4 carbons; wherein
X is a halogen;
R₁₈ is a H, or an alkyl chain up to 4 carbons;
n is 0, 1, 2, 3, 4,or5;
R₁₀ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group;
R₁₁ is a hydrogen, a halogen, acetamido, amino, pyridyl, a halo substituted pyridyl, a pyridyl salt, benzyl, 1- or 2-napthyl, styryl, phenethyl, furanyl, phenyl, a branched or straight-chained alkyl group up to 4 carbons, an ortho, meta, or para toluoyl, or a halo- or acetamido-substituted phenyl group; and
R₁₂, R₁₃, R₁₄, R₁₅, R₁₆, R₁₇ are a hydrogen, a halogen, trifluoromethyl, thioalkyl, alkoxy, hydroxy, amino, alkylamino, dialkylamino alkylamido, carbamyl alkyl ester, phenyl, or a branched or straight-chained alkyl group up to 4 carbons, with the proviso that when Z is N, R₁₂ is nonexistent,
or a compound having the structure of formula (I′″): 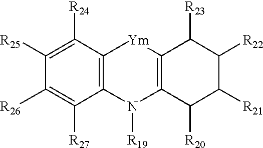
or a pharmaceutically acceptable salt or hydrate thereof, wherein:
Y is a S or O;
m is 0 or 1;
R₁₉ is an —(CH₂)ₙCO₂R₂₈, —(CH₂)ₙCHO, —(CH₂)ₙOH, —(CH₂)ₙCHOHCH₃, —(CH₂)ₙCHOHCH₂N(CH₃)₂, —(CH₂)ₙCHOHCH₂OH, —(CH₂)ₙC(OH)₂CX₃, —(CH₂)ₙCHOHCX₃, allyl, benzyl, H, or an alkyl chain up to 4 carbons;
X is a halogen; 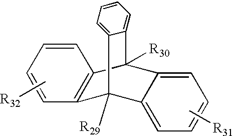
R₂₈ is a H, or any alkyl chain up to 4 carbons;
n is 0, 1, 2, 3, 4,or5;
R₂₀, R₂, are a hydrogen, or are part of a benzene ring;
R₂₂, R₂₃ are a hydrogen, or are part of a benzene ring; and
R₂₄, R₂₅, R₂₆, R₂₇ are a hydrogen, any halogen, trifluoromethyl, thioalkyl, alkoxy, alkylamido, phenyl, or a branched or straight-chained alkyl group up to 4 carbons.

107. The method of claim 106, wherein said azole is chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole, tioconazole, UR-9746, UR-9751, vibunazole, fluconazole, terconazole, genaconazole, itraconazole, voriconazole, posaconazole, ravuconazole, parconazole, SS 750, R120758, R-102557, T-8581, TAK 456, TAK 457, BMS 207147 or SYN 2869.

108. The method of claim 106, further comprising contacting said fungal cell with at least two second agents.

109. The method of claim 106, wherein said fungal cell is selected from the group consisting of Candida, Cryptococcus, Blastomyces, Histoplasma, Torulopsis, Coccidioides, Fusarium, Sporothorix, Paracoccidioides, Penicillium, Trichphyton, Microsporum, Pseudallescheria and Aspergillus.

110. The method of claim 106, wherein the triptycene has the formula (II′):
or a pharmaceutically acceptable salt or hydrate thereof, wherein:
R₂₉, R₃₀, R₃₁, R₃₂ are a H, —(CH₂)ₙCO₂R₃₃, —(CH₂)ₙOH, —(CH₂)ₙCHO, —(CH₂)ₙC(═O)CX₃, —(CH₂)ₙC(═O)Cl, —(CH₂)ₙC(═O)R₃₃, —(CH₂)ₙCHXR₃₃, —(CH₂)ₙCX₂R₃₃, —(CH₂)ₙN(R₃₃)₂, —(CH₂)ₙCH(OH)CH₃, —(CH₂)ₙCH(OH)CH₂OH, —CH═CHCH═CHCO₂R₃₃, —CH₂CH═CHCO₂R₃₃, —(CH₂)ₙCX₃, —C≡CCOR₄, —CON(R₃₃)₂, [1,3]-dioxolan-2-y phosphoryl dibenzylester, phosphoryl, sulfuryl, allyl, ethylene, nitro, or a halogen, wherein
X is a halogen;
R₃₃ is a H, or any alkyl chain up to 4 carbons; and
n=0, 1, 2, 3, 4,or5.

111. The method of claim 106, wherein the triphenyl has the formula (III): 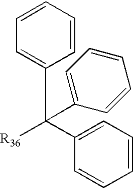
or a pharmaceutically acceptable salt or hydrate thereof, wherein:
R₃₆ is a H, —(CH₂)ₙCO₂R₃₇, —(CH₂)ₙOH, —(CH₂)ₙCHO, —(CH₂)ₙC(═O)CX₃, —(CH₂)ₙC(═O)Cl, —(CH₂)ₙN(R₃₇)₂, —(CH₂)ₙCH(OH)CH₃, —(CH₂)ₙCH(OH)CH₂OH, —CH═CHCH═CHCO₂R₃₇, —CH₂CH═CHCO₂R₃₇, —(CH₂)ₙCX₃, —C≡CCOR₃₇, —CON(R₃₇)₂, [1,3]-dioxolan-2-yl, phosphoryl dibenzylester, phosphoryl, sulfuryl, allyl, ethylene, nitro, or a halo; wherein
X is a halogen;
R₃₇ is a H, or any alkyl chain up to 4 carbons; and
n=0, 1, 2, 3, 4,or5.

112. The method of claim 106, wherein the compound having the structure of formula (I″) is a carbazole having the formula (I): 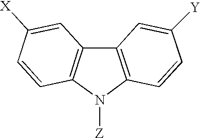
or a pharmaceutically acceptable salt or hydrate thereof, wherein:
X is an H, or a chloro, bromo, iodo, hydroxy, methoxy, amino, nitro, or dimethylamino;
Y is an H, or a chloro, bromo, iodo, hydroxy, methoxy, amino, nitro, or dimethylamino;
and
Z is —(CH₂)ₙCO₂H, —CH(OH)CH₃, —(CH₂)ₙCHOHCH₂OH, —(CH₂)ₙCH₂OH, —(CH₂)ₙC(═O)(CH₂)ₘCH₃, or —(CH₂)ₙCO₂(CH₂)ₘCH₃, wherein n and m are independently 0, 1, 2, 3,
or Z is 4, 3-(4-styryl-piperazin-1-yl)propan-2-ol, an alkyl amide, or an alkoxyl.

113. The method of claim 106, wherein contacting the fungal cell with a second agent can occur before, during, or after azole administration.
